(19) United States
(12) Patent Application Publication
(10) Pub. No.: US 2025/0155774 A1
(43) Pub. Date: May 15, 2025

(54) NONLINEAR SOLID STATE DEVICES FOR OPTICAL RADIATION IN FAR-UV SPECTRUM

(71) Applicant: Uviquity, Inc., Raleigh, NC (US)

(72) Inventors: Brent Fisher, Bethesda, MD (US); Scott Burroughs, Raleigh, NC (US); Russell Kanjorski, Chapel Hill, NC (US)

(21) Appl. No.: 18/837,695

(22) PCT Filed: Feb. 16, 2023
(86) PCT No.: PCT/US2023/013187

§ 371 Date: Aug. 12, 2024

Related U.S. Application Data
(60) Provisional application No. 63/359,251, filed on Jul. 8, 2022.
(60) Provisional application No. 63/311,660, filed on Feb. 18, 2022.

(51) Int. Cl. G02F 1/35; G02F 1/39; H01S 5/00; H01S 5/06

(52) U.S. Cl. CPC G02F 1/3532; G02F 1/3544; G02F 1/39; H01S 5/0092; H01S 5/0605

(57) ABSTRACT

An ultraviolet (UV) light source includes a light emitting element that is configured to generate light of a first frequency, a nonlinear optical element that is configured to receive the light of the first frequency from the light emitting element and generate UVC or far-UVC light of a second frequency from the light of the first frequency, and an output coupling element that is configured to selectively outcouple the UVC or far-UVC light from the nonlinear optical element as output light. Related light sources and fabrication methods are also discussed.

### CLAIM OF PRIORITY

The present application claims priority from U.S. Provisional Patent Application No. 63/311,660 filed Feb. 18, 2022, and U.S. Provisional Patent Application No. 63/359,251 filed Jul. 8, 2022, with the United States Patent and Trademark Office, the disclosures of which are incorporated by reference herein in their entireties.

### FIELD

The present application is directed to UV light sources, and in particular, to far-UVC light sources and related devices and methods.

### BACKGROUND

Compact and efficient ultraviolet (UV) light sources in the wavelength range of about 200 nanometers (nm) to about 400 nm may be desirable for many applications. For example, UV lasers may be used for lithography in semiconductor manufacturing. Since short-wavelength radiation is easily absorbed by most materials, another application is the detection and classification of materials and substances, such as in mass spectroscopy. Photons in the UV-C (or UVC) wavelength range (e.g., about 200 nm to about 280 nm) can be used to disinfect airborne and surface disease-causing pathogens while remaining safe for human exposure. For example, far-UVC light (from about 200 nm to about 240 nm) may not penetrate through the dead-cell layer of the skin surface or the tear layer of the human eye, but may be effective against bacteria and viruses. In particular, far-UVC light can efficiently cause permanent physical damage to DNA, which can prevent bacteria, viruses and fungi from replicating. Human-safe far-UVC light can thus effectively kill disease causing pathogens with little to no risk to humans because these wavelengths may be largely absorbed by the stratum corneum (the top layer of dead skin cells in the epidermis).

However, operation in the far-UVC wavelength range may present challenges. For example, few available light sources may be configured for operation in the far-UV. Some conventional UV light sources have been implemented by gas-based lamps. An important class of such lamps is called “excimer” (excited dimer) lamps that employ a mixture of a reactive gas (such as F₂ or Cl₂) and an inert gas (such as Kr, Ar or Xe) as an active medium. The gas mixtures, when electrically excited, produce a pseudo-molecule excited state dimer, or ‘excimer’ with an energy level configuration that allows the generation of specific ultraviolet laser wavelengths. For example, some KrCl lamps may be used to generate Far UVC light for medical applications. However, the inefficiency, large size, and significant cost of such lasers may be prohibitive for use in many applications.

Also, high power, ultrafast laser systems designed for laboratory use can generate non-linear harmonics (e.g., second, third, fourth, and fifth harmonic generation) or parametric sum frequency generation to create light in the far-UV. Such systems may likewise be large (e.g., table-top size or macroscopic optical bench size), expensive, and inefficient (e.g., generating less than a watt optical in the far UV).

Free-electron pumped field emission lamps with hexagonal Boron Nitride (h-BN) target may rely on bulbs that are vacuum sealed to allow the electron beam to operate, but the power efficiency and reliability of such lamps may be unproven.

Semiconductor-based LED light sources (e.g., based on GaN material system) have also been used to provide UV-C light, for example, using phosphor-based wavelength conversion. Such light sources typically have short operating lifetimes and poor performance at emission wavelengths shorter than about 265 nm. Also, due to residual uncertainty about human safety, regulatory limits remain strict.

### SUMMARY

According to some embodiments, an ultraviolet (UV) light source includes a light emitting element that is configured to generate light of a first frequency, a nonlinear optical element that is configured to receive the light of the first frequency from the light emitting element and generate far-UVC light of a second frequency from the light of the first frequency, and an output coupling element that is configured to selectively outcouple the far-UVC light from the nonlinear optical element as output light.

In some embodiments, the output coupling element is configured to selectively outcouple the far-UVC light into at least one direction that is different than a direction of propagation of the light of the first frequency to provide the output light.

In some embodiments, the output light is substantially free of the light of the first frequency.

In some embodiments, the nonlinear optical element, the light emitting element, and/or the output coupling element comprise elements of a same material system. In some embodiments, the nonlinear optical element comprises aluminum nitride (AlN). In some embodiments, the light emitting element and/or the output coupling element comprise a Group III nitride-based material.

In some embodiments, the nonlinear optical element is or comprises an optical cavity that is at least partially resonant at the first frequency.

In some embodiments, the nonlinear optical element has a ring configuration that defines the optical cavity.

In some embodiments, the nonlinear optical element comprises a plurality of nonlinear optical elements that are arranged to receive the light of the first frequency from the light emitting element.

In some embodiments, an input coupling element is configured to receive the light of the first frequency from the light emitting element, and the plurality of nonlinear optical elements are arranged along the input coupling element.

In some embodiments, respective ones of the nonlinear optical elements comprise different dimensions and/or materials. The output coupling element comprises a plurality of output coupling elements that are respectively configured to selectively outcouple the far-UVC light from the respective ones of the nonlinear optical elements.

In some embodiments, the optical cavity includes the light emitting element and the nonlinear optical element therein.

In some embodiments, the optical cavity has a linear shape or a closed curve shape.

In some embodiments, output coupling element comprises at least one of a facet having a refractive index that is configured to selectively outcouple the far-UVC light in a first direction, or a grating having a diffraction order that is configured to selectively outcouple the far-UVC light in a second direction, different than the first direction.

In some embodiments, the nonlinear optical element and the output coupling element are integrated in an output element that is configured to outcouple the far-UVC light at a plurality of positions or continuously along a length thereof.

In some embodiments, the UV light source is configured to provide the output light substantially free of phase matching between the light of the first frequency and the far-UVC light of the second frequency.

In some embodiments, at least one of the nonlinear optical element and the output coupling element is configured to provide phase matching between the far-UVC light of the second frequency and the light of the first frequency.

In some embodiments, the light emitting element is a laser comprising a lasing cavity. The laser is configured to generate the light of the first frequency. In some embodiments, the laser comprises a Group Ill nitride-based material.

In some embodiments, the light emitting element further comprises one or more optical resonators that are configured to reflect the light of the first frequency and are arranged at first and second ends of the lasing cavity.

In some embodiments, the nonlinear optical element is configured to receive the light of the first frequency from an intra-cavity portion between first and second ends of the lasing cavity.

In some embodiments, the nonlinear optical element comprises first and second nonlinear optical elements positioned at first and second ends of the lasing cavity, respectively.

In some embodiments, a saturable absorber in the lasing cavity is configured to generate the light of the first frequency as a plurality of light pulses at a predetermined pulse repetition frequency and duty factor.

In some embodiments, at least one tuning mechanism is configured to adjust one or more operating characteristics of the nonlinear element based on the light of the first frequency.

In some embodiments, a monitor element is configured to measure a property of the output light and generate a feedback signal to a controller that is configured to operate the light emitting element and/or the tuning mechanism.

In some embodiments, a substrate includes the light emitting element, the nonlinear optical element, and the output coupling element on a surface thereof, where two or more of the light emitting element, the nonlinear optical element, the output coupling element, or connecting waveguides therebetween overlap in a direction perpendicular to the surface of the substrate.

In some embodiments, the output coupling element comprises a plurality of output coupling elements that are configured to outcouple the far-UVC light in respective directions, to provide the output light with a desired far field pattern.

In some embodiments, one or more sensors are configured to detect real-time conditions in an operating environment of the UV light source, and to transmit detection signals indicating the real-time conditions to a controller that is configured to control operation of the light emitting element based on the detection signals.

In some embodiments, the second frequency comprises a sum of or a harmonic of the first frequency.

In some embodiments, the first frequency corresponds to a first wavelength in a range of about 400 nanometers (nm) to 480 nm, and the second frequency corresponds to a second wavelength in a range of about 200 nm to 240 nm.

In some embodiments, the light emitting element and the nonlinear optical element comprise respective elements that are arranged on a non-native substrate.

In some embodiments, the light emitting element and the nonlinear optical element are integrated in a monolithic structure.

In some embodiments, the UV light source comprises an array including a plurality of the light emitting element and the nonlinear optical element.

According to some embodiments, a light source includes a monolithic structure comprising a light emitting element that is configured to generate light of a first frequency, and a nonlinear optical element that is configured to receive the light of the first frequency from the light emitting element and generate light of a second frequency from the light of the first frequency.

In some embodiments, the monolithic structure further comprises an output coupling element that is configured to selectively outcouple the light of the second frequency from the nonlinear optical element as output light. The output coupling element is configured to selectively outcouple the light of the second frequency into at least one direction that is different than a direction of propagation of the light of the first frequency to provide the output light.

In some embodiments, the nonlinear optical element of the monolithic structure comprises aluminum nitride (AlN). In some embodiments, the light emitting element and/or the output coupling element of the monolithic structure comprise a Group III nitride-based material.

In some embodiments, the light emitting element is a laser comprising a lasing cavity, and the nonlinear optical element is configured to receive the light of the first frequency from an intra-cavity portion between first and second ends of the lasing cavity.

In some embodiments, the light of the second frequency is UVC light, such as far-UVC light. In some embodiments, the light of the first frequency is visible light.

According to some embodiments, an ultraviolet (UV) light source includes a light emitting element that is configured to generate light of a first frequency, and a nonlinear optical element comprising aluminum nitride (AlN) that is configured to receive the light of the first frequency from the light emitting element and generate UVC light of a second frequency from the light of the first frequency.

In some embodiments, an output coupling element is configured to selectively outcouple the UVC light from the nonlinear optical element as output light, in some embodiments into at least one direction that is different than a direction of propagation of the light of the first frequency. The light emitting element and/or the output coupling element may include a Group III nitride-based material, in some embodiments in a monolithic structure.

According to some embodiments, an ultraviolet (UV) light source includes a light emitting element that is configured to generate light of a first frequency, and an optical cavity comprising a nonlinear optical element that is configured to receive the light of the first frequency from the light emitting element and generate UVC light of a second frequency from the light of the first frequency, where the optical cavity is at least partially resonant at the first frequency.

In some embodiments, the optical cavity is at least partially resonant at the first frequency and at the second frequency.

In some embodiments, the optical cavity comprises a plurality of optical cavities, each comprising a respective nonlinear optical element and arranged to receive the light of the first frequency from the light emitting element. In some embodiments, the optical cavities are ring-shaped.

In some embodiments, respective ones of the optical cavities include different dimensions and/or materials, and the output coupling element comprises a plurality of output coupling elements that are respectively configured to selectively outcouple the UVC light from the respective ones of the optical cavities.

In some embodiments, the nonlinear optical element and the output coupling element are integrated in an output element that is configured to outcouple the UVC light at a plurality of positions or continuously along a length thereof.

According to some embodiments, a light source includes a light emitting element that is configured to generate light of a first frequency, and a nonlinear optical output coupling element that is configured to receive the light of the first frequency from the light emitting element, generate light of a second frequency from the light of the first frequency, and outcouple the light of the second frequency as output light at a plurality of positions or continuously along a length thereof.

In some embodiments, the light source is configured to provide the output light substantially free of phase matching between the light of the first frequency and the light of the second frequency.

In some embodiments, the nonlinear optical output coupling element is configured to selectively outcouple the light of the second frequency into at least one direction that is different than a direction of propagation of the light of the first frequency to provide the output light.

In some embodiments, the nonlinear optical output coupling element includes or is coupled to an optical cavity that is at least partially resonant at the first frequency.

In some embodiments, the nonlinear optical output coupling element comprises a plurality of alternating nonlinear optical element sections and output coupling element sections along the length thereof.

In some embodiments, the nonlinear optical output coupling element comprises first and second materials that are configured to alter light propagation at one of a first wavelength corresponding to the first frequency and a second wavelength corresponding to the second frequency, and do not substantially alter light propagation at another of the first wavelength and the second wavelength.

In some embodiments, the nonlinear optical output coupling element is a waveguide comprising nanopores or defects therein having respective dimensions that are configured to scatter the light of the second frequency, without substantially affecting propagation of the visible light of the first frequency.

In some embodiments, the first frequency corresponds to a first wavelength in a range of about 400 nanometers (nm) to 480 nm, and the second frequency corresponds to a second wavelength in a range of about 200 nm to 240 nm.

Other devices, apparatus, and/or methods according to some embodiments will become apparent to one with skill in the art upon review of the following drawings and detailed description. It is intended that all such additional embodiments, in addition to any and all combinations of the above embodiments, be included within this description, be within the scope of the invention, and be protected by the accompanying claims.

### BRIEF DESCRIPTION OF THE DRAWINGS

FIG. 1A is a schematic block diagram illustrating a UV light source according to some embodiments of the present disclosure.

FIG. 1B is a schematic block diagram illustrating elements of a UV light source according to some embodiments of the present disclosure in greater detail.

FIG. 1C is a graph illustrating an emission range for light output from a UV light source according to some embodiments of the present disclosure.

FIGS. 2A1 and 2A2 are schematic perspective and side views, respectively, illustrating elements of a UV light source in a vertical linear arrangement according to some embodiments of the present disclosure.

FIGS. 2B1 and 2B2 are schematic perspective and top views, respectively, illustrating elements of a UV light source in a horizontal linear arrangement according to some embodiments of the present disclosure.

FIGS. 2C1 and 2C2 are schematic top views illustrating elements of a UV light source in a spiral arrangement according to some embodiments of the present disclosure.

FIGS. 3A1 and 3A2 are schematic block diagrams illustrating elements of a UV light source including optical cavity enhancement according to some embodiments of the present disclosure.

FIGS. 3B1 and 3B2 are schematic perspective and top views, respectively, illustrating elements of a UV light source including optical cavity enhancement in a horizontal linear arrangement according to some embodiments of the present disclosure.

FIG. 4A is a schematic block diagram illustrating elements of a UV light source including a nonlinear optical element in a ring cavity configuration according to some embodiments of the present disclosure.

FIG. 4B is a schematic top view illustrating elements of a UV light source including a plurality of nonlinear optical elements in ring cavity configurations that are sequentially arranged according to some embodiments of the present disclosure.

FIG. 4C1 is a schematic top view illustrating elements of a UV light source including a nonlinear optical element coupled to an intra-cavity portion of the light emitting element according to some embodiments of the present disclosure.

FIG. 4C2 is a schematic top view illustrating an array of UV light sources that respectively include a nonlinear optical element coupled to an intra-cavity portion of the light emitting element according to some embodiments of the present disclosure.

FIG. 4C3 is a graph illustrating vernier frequency selection for determining optical cavity size (including height, width, and circumference/length) of a ring-shaped nonlinear optical element according to some embodiments of the present disclosure.

FIG. 5A is a schematic block diagram illustrating elements of a UV light source in which the light emitting element and the nonlinear optical element are provided in a same optical cavity according to some embodiments of the present disclosure.

FIGS. 5B1 and 5B2 are schematic perspective and top views, respectively, illustrating elements of a UV light source in a same optical cavity with an output coupling element implemented as a reflective facet configured for selective light extraction in a horizontal linear arrangement according to some embodiments of the present disclosure.

FIGS. 5C1 and 5C2 are schematic perspective and top views, respectively, illustrating elements of a UV light source in a same optical cavity with an output coupling element implemented as an optical grating configured for selective light extraction in a horizontal linear arrangement according to some embodiments of the present disclosure.

FIG. 6A is a schematic block diagram illustrating elements of a UV light source in which the light emitting element and the nonlinear optical element are provided in a same optical cavity in a closed curve or racetrack configuration according to some embodiments of the present disclosure.

FIGS. 6B1 and 6B2 are schematic perspective and top views, respectively, illustrating elements of a UV light source in a same optical cavity in a closed curve or racetrack configuration with an output coupling element implemented as an optical grating configured for selective light extraction according to some embodiments of the present disclosure.

FIGS. 7A, 7B, and 7C illustrate example drive signals to provide pulsed light output from light emitting elements in a UV light source according to some embodiments of the present disclosure.

FIG. 8 is a schematic perspective view illustrating respective elements of a UV light source arranged in a hybrid configuration on a common non-native substrate according to some embodiments of the present disclosure.

FIG. 9 is a schematic block diagram illustrating an array of UV light sources that respectively include a light emitting element and a nonlinear optical element according to some embodiments of the present disclosure.

FIGS. 10A and 10B are schematic perspective and top views, respectively, illustrating elements of a UV light source including optical cavity enhancement with an output coupling element configured to provide distributed emission and selective light extraction in a horizontal linear arrangement according to some embodiments of the present disclosure.

FIG. 11A is a schematic top view illustrating an example combination of various elements of a UV light source including a nonlinear optical element coupled to an intra-cavity portion of a light emitting element, in combination with ring resonators at respective ends of the light emitting element and an output coupling element configured for selective light extraction according to some embodiments of the present disclosure.

FIG. 11B are graphs illustrating a vernier frequency effect according to some embodiments of the present disclosure that provides for selection of a subset of possible longitudinal modes supported by the light emitting element.

FIG. 12 is a schematic top view illustrating an example combination of various elements of a UV light source including nonlinear optical elements coupled to an extra-cavity portions of a light emitting element, in combination with ring resonators at respective ends of the light emitting element, tuning mechanisms, output coupling elements configured for selective light extraction, and an output monitor according to some embodiments of the present disclosure.

FIG. 13 is a schematic top view illustrating an example combination of various elements of a UV light source including nonlinear optical elements coupled to an extra-cavity portions of a light emitting element including a saturable absorber element, in combination with tuning mechanisms, output coupling elements configured for selective light extraction, and an output monitor according to some embodiments of the present disclosure.

FIG. 14 is a schematic top view illustrating an example combination of various elements of a UV light source including a plurality of different or non-identical nonlinear optical elements 120 in ring cavity configurations with respective output coupling elements according to some embodiments of the present disclosure.

FIG. 15 is a schematic top view illustrating an example combination of various elements of a UV light source in physically overlapping configurations according to some embodiments of the present disclosure.

FIG. 16 is a schematic top view illustrating including output coupling element configurations of a UV light source to provide a desired far field emission pattern according to some embodiments of the present disclosure.

FIG. 17 is a schematic block diagram illustrating components of a sensor feedback-based “smart” illumination device that includes a germicidal UV (GUV) light source communicatively coupled to sensors that are configured to feedback information to a controller of the GUV light source according to some embodiments of the present disclosure.

### DETAILED DESCRIPTION

Embodiments of the present disclosure provide solutions for generating electromagnetic radiation in the far-UVC wavelength band (about 200 nm to 240 nm, for example, about 207 nm to 222 nm) and related control of illumination patterns, which can be useful for numerous applications, including (but not limited to) germicidal applications for disinfecting airborne and surface disease-causing pathogens, and detection of trace chemical or biological species in various field environments (air, water, etc.), while simultaneously remaining safe for human exposure and complying with human safety regulations and requirements. In particular, embodiments of the present disclosure provide a solid state system and method for generating coherent or non-coherent, collimated or non-collimated, electromagnetic, non-ionizing radiation in the far-UVC wavelength band, based on nonlinear optical processes and using photonic integrated circuits (PIC). As used herein, “far-UV” or “Far UV” wavelength band or range refers to wavelengths greater than about 200 nm (such that the radiation is non-ionizing in the atmosphere), and less than about 240 nm, for example, about 200 nm to 230 nm.

Embodiments of the present disclosure allow generation of light in the far-UVC band using compact sources based on materials and processes from the semiconductor industry which will allow rapid volume scaling reduction of cost that may not be available by other methods. Embodiments of the present disclosure may provide a far-UVC light source including a semiconductor light emitting element, such as a pump laser (e.g. a Group-III nitride-based laser diode) configured to generate light of a first wavelength (e.g., in the visible spectrum, also referred to herein as visible light), which is coupled into a nonlinear optical element (e.g., a monolithically integrated waveguide with nonlinear optical properties) for generation of light that is a sum of the frequency of the visible light (also referred to herein as sum frequency generation (SFG), e.g., Second Harmonic Generation (SHG) of frequency doubled light). Sum frequency generation may include both frequency doubling (combination of photons of the same wavelength) and optical parametric conversion (i.e., from combination or difference of two photons of unequal wavelength). The nonlinear optical element may be referred to herein as an SHG element, or more generally, an SFG element. The sum frequency generation or frequency-doubling converts a portion of the visible light emitted by the light emitting element into far-UVC light. In particular, some embodiments of the present disclosure provide a monolithic, solid-state Far UV Photonic Integrated Circuit (PIC) (for example, based upon the AlN/GaAlN material system), which may be scalable to high volumes, low cost, high WPE, and small form factors without the need for an optical filter that discriminates or transmits light only within a range of far-UVC wavelengths.

As used herein, the term monolithic structure or monolithic integration may refer to any arrangement of active elements (e.g., light emitting elements) and/or passive elements (e.g., waveguides or other optical coupling elements) in a unitary structure with no air interfaces or free propagation of light between elements, including structures formed by epitaxial growth, wafer bonding, and/or microtransfer printing or other forms of mass transfer for solid state integration. Monolithic integration may thus include elements of the same material system or multiple materials, and may be provided on a native (e.g., growth) substrate or on a non-native substrate (which is different from the native or source substrate on which the elements are grown or otherwise formed). In contrast, a hybrid structure or hybrid integration may refer to arrangement of separate or discrete elements (e.g., respective semiconductor chips) with air interfaces between elements and/or assembly of such discrete elements on a non-native substrate. Elements that are “coupled” may refer to physical and/or optical coupling.

U.S. Pat. No. 9,159,178 describes the use of a semiconductor diode laser as the pump frequency which has a single pass through a non-linear crystal (BBO) and is critically phase matched by means of angle tuning. The publication “Periodically-Poled AlN for frequency doubling” to Sitar et al. describes AlN as a nonlinear material, but seeks to achieve phase matching by periodically poling the AlN, with a macroscopic pump laser that is externally coupled into the AlN ridge waveguide with major optical losses.

Second (or third, fourth, etc.) harmonic frequency generation using nonlinear optical materials in accordance with some embodiments may require several components or characteristics for efficient conversion. For example, a nonlinear crystal that is non-centrosymmetric and highly polarizable may lead to non-zero elements of its second order nonlinearity tensor, where the higher this coefficient, the higher the conversion rate. The nonlinear crystal should be optically transparent at the wavelength of the frequency doubled light; otherwise the crystal would absorb the newly generated light. Also a pump light source that is coherent (second harmonic generation is a coherent effect relevant to a single wavelength, so the pump laser may have a narrow linewidth with sufficiently long coherence length) and high power (the output power of second harmonic generation scales with the square of the pump power; therefore the higher the power of the pump laser the higher the efficiency of conversion) may be used. In some embodiments, pulsed lasers, which generally have higher peak pulse power than continuous wave (CW) lasers, may be preferred. In some embodiments, phase matching methods may be used to match the phase speed of the pump wavelength to that of the second harmonic wavelength such that coherent addition of the electric field from both waves is maintained over the entire propagation length of the nonlinear crystal.

According to some embodiments of the present disclosure, a UV light source includes a light emitting element (e.g., a Group-III nitride-based laser diode, such as a blue pump laser diode) configured to generate light of a first (fundamental) frequency or wavelength (e.g., visible light), a nonlinear optical element (e.g., a nonlinear optical crystal, such as a SHG element) that is optically transparent to wavelengths at or below the desired output wavelength (e.g., the UVC wavelength range of about 200 nm to about 280 nm, or the far-UVC wavelength range of about 200 nm to about 240 nm) and is configured to generate UVC or far-UVC light of a second frequency or wavelength based on sum frequency generation of the light of the first frequency; an input coupling element configured to couple the light from the light emitting element into the nonlinear optical element (e.g., a continuous waveguide or optical fiber or a photonic wirebond that connects radiation from the pump laser to the nonlinear optical crystal; also referred to herein as an input waveguide); and an output coupling element configured to selectively outcouple the UVC or far-UVC light from the nonlinear optical element. In some embodiments, one or more elements may provide phase matching between the UVC or far-UVC light of the second frequency or wavelength and the fundamental (pump) frequency or wavelength of the visible light.

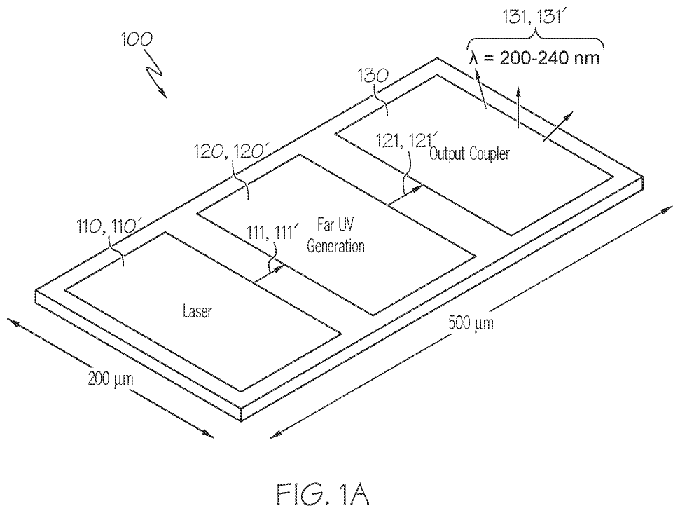

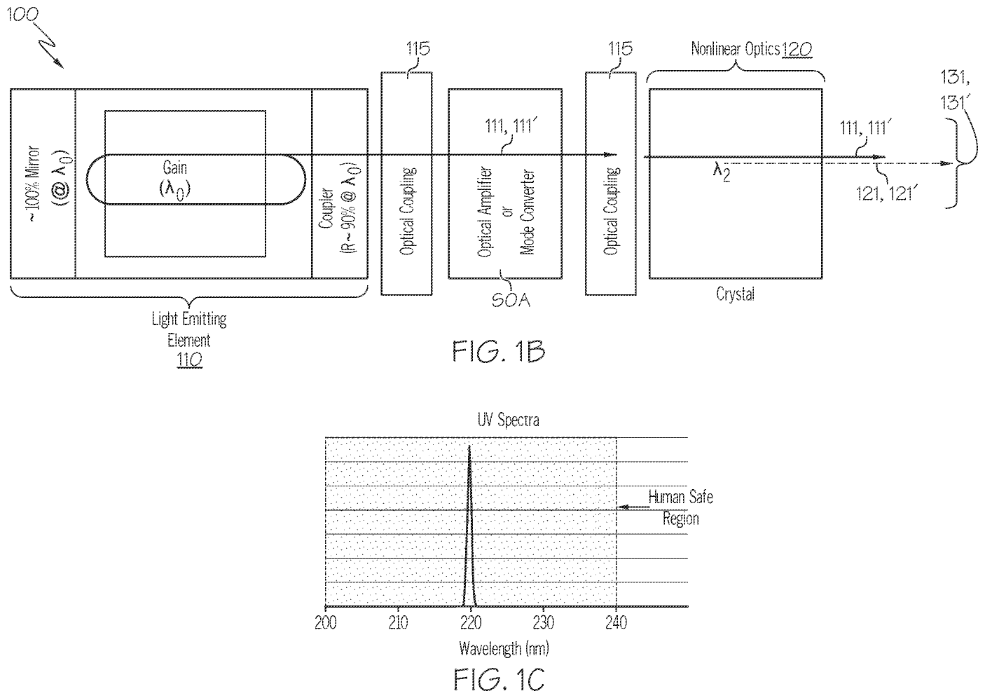

FIG. 1A is a schematic block diagram illustrating a UV light source 100 according to some embodiments of the present disclosure. As shown in FIG. 1A, an ultraviolet (UV) light source 100 includes a light emitting element 110, 110′ that is configured to generate light 111 (e.g., visible light 111′) of a first frequency, a nonlinear optical element 120, 120′ that is configured to receive the light 111, 111′ from the light emitting element 110 and generate light 121 (e.g., far-UVC light 121′) of a second frequency based on sum frequency generation of the light 111, 111′ of the first frequency, and an output coupling element 130 that is configured to selectively outcouple the light 121, 121′ from the nonlinear optical element 120, 120′ as output light 131, 131′. While described herein primarily with reference to generation of far-UVC light (including wavelengths within about 200 nm to about 240 nm), it will be understood that embodiments of the present disclosure are not limited to generations of far-UVC light. For example, some embodiments may include nonlinear optical elements 120 configured to generate light over a wider wavelength range, such as UVC light (including wavelengths within about 200 nm to about 280 nm).

In some embodiments, the light emitting element 110 may be a blue pump laser 110′ that produces (high power) coherent radiation at wavelengths between about 400 nm to about 460 to 480 nm with good wall plug efficiency (optical power output per unit electrical power consumption). In some embodiments, the laser 110′ may be a laser diode (for example, an edge emitting laser or a vertical-cavity surface-emitting laser (VCSEL)). However, other lasers (for example, a frequency doubled fiber laser) may also be used. The light emitting element 110 may be formed of or otherwise include a Group-III nitride-based material (such as gallium nitride (GaN)).

The nonlinear optical element 120 may be configured to generate far-UVC light 121′ of a second frequency based on sum frequency generation of the light of a first frequency that is output from the light emitting element 110. The second frequency may be a harmonic (e.g., integer multiple) of the first frequency. The nonlinear optical element 120 may be a nonlinear optical crystal that is optically transparent to wavelengths at or below the far-UVC output wavelength of about 200 to 240 nm. Examples of such nonlinear optical crystals may include, but are not limited to, BBO, aluminum nitride (AlN), lithium niobate (LiNbO₃), etc.

AlN is not (to the inventors' knowledge) generally used to provide nonlinear optical elements. Rather, those of skill in the art in the field of nonlinear optics (as distinct from those of skill in the art in the field of semiconductor processing) have typically relied on bulk crystals, for example, using angle tuning of birefringent materials to achieve phase matching. Common suppliers of such bulk nonlinear crystals likewise have not recognized and do not sell AlN nonlinear optics. Also, bulk crystalline AlN may not be well suited for operation in the UV wavelength ranges due to a large quantity of point defects therein, which can absorb light having shorter wavelengths.

While thin-film AlN may be optically transparent to wavelengths of light as short as 200 nm, such thin films of AlN have been limited to use of waveguides. Research using thin-film AlN for nonlinear optical conversion has typically been limited to longer wavelengths of light, for example, due to some inherent challenges with achieving similar results at shorter wavelengths of light. For instance, polycrystalline thin films fabricated by sputtering may include grain boundaries that create absorption and scattering at short wavelengths of light (similar to point defects in bulk AlN). Fabrication of PICs in AlN with the fidelity required for acceptable nonlinear conversion may likewise be difficult at shorter wavelengths of light. Such challenges may manifest as losses in the nonlinear optical element, and thus may present barriers to realizing desired performance at short wavelengths. In particular, fabrication fidelity may present difficulties in achieving sufficient phase matching.

Some embodiments of the present disclosure may arise from realization that delivery of higher intensity pump laser light into the nonlinear optical element may overcome the aforementioned optical loss challenges. Embodiments of the present disclosure provide various configurations for obtaining higher optical intensity inside the nonlinear optical element, including (but not limited to) monolithic integration, cavity enhancement, and intra-cavity-tapping as described herein. Embodiments of the present disclosure also address challenges with respect to phase matching, which may be more difficult at shorter wavelengths.

AlN may be advantageous for nonlinear optical element 120 formation (e.g., by epitaxial growth) in combination with a Group III nitride-based light emitting element 110 material, such as GaN, due to lattice compatibility or similarity of material processing to that of GaN. More generally, the nonlinear optical element 120 and the light emitting element 110 may include common elements or materials that belong to the same material system (e.g., AlN may be used as a nonlinear optical element 120′ because it belongs to the same AlGaInN material system from which a GaN light emitting element 110′ is formed). Particular embodiments are described herein with reference to AlN-based nonlinear optical elements 120 (and in some embodiments, with reference to light sources where the light emitting element 110, the nonlinear optical element(s) 120, and the coupling elements 115, 130 are all nitride-based materials), but are not limited thereto.

The output coupling element 130 may refer to an optical element that is configured to provide the output light 131 (e.g., the far-UVC light 121′) for propagation through free space. In some embodiments, the output coupling element 130 may be configured to provide selective light extraction, such that the output light 131 may include primarily the far-UVC light 121′ of the second wavelength or frequency (and in some instances, may be substantially free of the visible light 111′ of the first wavelength or frequency), in one or more directions that differ from the direction(s) of propagation of the light 111 of the first wavelength or frequency. The output coupling element 130 may be implemented as part of a waveguide (also referred to herein as an output waveguide) and/or an edge facet in some embodiments. In some embodiments, the output coupling element 130 may be a grating for generating the different direction(s) of propagation of the output light 131 as surface emission, such as surface normal (or near surface normal) emission. In some embodiments, the output coupling element 130 may be integrated with the nonlinear optical element 120 (also referred to herein as a nonlinear optical output coupling element 120/130).

FIG. 1B is a schematic block diagram illustrating elements of a UV light source according to some embodiments of the present disclosure in greater detail. As shown in FIG. 1B, the UV light source includes the light emitting element 110 (shown as pump laser 110′ configured to output visible light 111′ having a wavelength λ₀ between 400 nm and 480 nm) and the nonlinear optical element 120 (shown as a SFG element configured to output light 121′ having a wavelength λ₂ between 200 nm and 240 nm, e.g., ½λ₀). Some subcomponents of the laser 110′ include the gain medium and the optical structures which form an optical cavity 125. The gain medium is pumped (electrically, optically, and/or by other means) to achieve population inversion, and the “pump light” 111 (used herein to refer to the output of the laser 110′ or other light emitting element 110) experiences cavity effects and propagates through elements. In the example of FIG. 1B, a simple Fabry Perot cavity is shown with an asymmetry in the reflectivity, such that emission is favored on one side. This embodiment illustrates a single pass of the pump light 111 through the SFG element, generating SFG light (λ₂) along the way, but other embodiments may employ optical cavity 125 at either (λ₀ or λ₂ or both) to generate resonant enhancement of the SFG element. That is, it is understood that this example is by way of illustration only, and may be used with other semiconductor diode lasers including (but not limited to) edge emitters with cleaved facet end mirrors, distributed feedback grating structures or photonic crystal structures for generating the optical cavity 125. The example illustration may also represent a vertical cavity surface emitting laser (VCSEL) in which the gain is a multi-quantum well (MQW) structure and the end mirrors are dielectric Bragg reflectors (DBRs). Optional active or passive elements, such as optical amplifiers, mode converters, etc., may be provided at the output of the light emitting element 110 to enhance performance in some embodiments.

The input coupling element 115 is configured to couple the visible light 111′ from the light emitting element 110 into the nonlinear optical element 120. The input coupling element 115 may be a continuous waveguide that connects radiation from the pump laser to the nonlinear optical crystal. Some embodiments may include optical coupling by non-waveguide means, for example, free space propagation and focusing with lenses; optical fibers; etc. That is, the input optical coupling element may be implemented by any optical element that is configured to relay the light output from the light emitting element 110 to the nonlinear optical element 120.

In some embodiments, the UV light source may be configured to provide phase matching between the second frequency ω2 or wavelength λ₂ of the far-UVC light 121′ generated by the nonlinear optical element 120 (also referred to herein as the SHG or SFG wavelength or wavelength range) and first frequency ω₁ or wavelength λ₀ of the visible light 111′ generated by the light emitting element 110 (also referred to herein as the fundamental or pump wavelength or wavelength range). For example, the phase matching may be provided by implementing the nonlinear optical element 120 as a waveguide (which may or may not have optical resonance) that is configured such that the speed of propagation modes supported at fundamental and SHG/SFG wavelengths are identical and thus phase matched. However, in some embodiments other means of phase matching may be used, such as (but not limited to) periodically poled crystals (i.e., quasi phase matching, type 0) or birefringence in the nonlinear crystal for type 1 or type 2 phase matching. In other embodiments, the UV light source may be substantially free of phase matching (i.e., may be configured to provide the output light without phase matching methods or with relaxed phase matching requirements to match propagation of the visible light 111′ of the first frequency with the far-UVC light 121′ of the second frequency). For example, some embodiments may provide distributed, selective outcoupling of the SFG/SHG light 121′ such that light of the second frequency does not propagate long distances in the waveguide and thus allows the device to achieve satisfactory performance instead of or in the absence of phase matching.

FIG. 1C is a graph illustrating an emission range for light output from a UV light source according to some embodiments of the present disclosure. As shown in FIG. 1C, the output light 131 includes far-UVC light 121′ having a wavelength in a range of about 200 nm to 240 nm, for example, about 207 nm or 222 nm, and may or may not include the visible light 111′. UV light sources in accordance with embodiments of the present disclosure may lack requirements as to the nature or quality of the emitted SFG (e.g., far-UV) light. Any generation of photons at the required wavelength in sufficient quantity to meet the purposes of the intended application may be used in embodiments as described herein. In other words, some embodiments may operate without specific requirements on the beam quality of the SFG light, or may not require a beam at all. The emitted SFG light can diverge, can be unpolarized, and/or can scatter, Embodiments described herein thus span producing light of all levels of quality (e.g., coherent or non-coherent, collimated or non-collimated) so long as the wavelength is contained in a well-defined band (e.g., the far-UV) based on second (or higher) harmonic generation from a coherent pump laser.

Referring again to FIG. 1A, embodiments of the present disclosure may monolithically integrate active optical components (such as the light emitting element 110) and passive optical components (such as nonlinear optical elements 120, low loss optical waveguides, resonant cavities, optical couplers, etc.) onto a single chip (e.g., a single PIC) that is configured to generate human-safe far-UVC light 121′, without free-space propagation of the light between elements or components. Monolithic integration may be advantageous by reducing losses in coupling of light from the active element 110 (e.g., laser 110′) to the passive element 120 (e.g. SHG/SFG element 120′). As used herein, a “die” or chip may refer to a small block or body of semiconducting material or other substrate on which elements are fabricated, for example, to provide monolithic integration of active and passive optical elements. In other embodiments, the active and passive optical elements may be respective elements that are arranged or assembled on a non-native substrate. Such hybrid integration may include embodiments where the light emitting element 110, the nonlinear optical element 120, and/or other passive components are separately packaged or are on separate dies, and optical coupling is implemented by fiber or free space propagation.

In some embodiments, the output coupler(s) may be configured such that the desired wavelength of light (e.g., 220 nm) is preferentially supported and outcoupled (rather than the fundamental frequency or wavelength of the laser 110′, e.g., 440 nm), referred to herein as selective light extraction or selective outcoupling. However, it will be understood that selective outcoupling of the far-UVC light 121 does not require an absence of the undesired wavelengths of light (e.g., the pump light 111) in the output light 131. For example, because the optical power of the visible light 111′ may be an order of magnitude stronger than the SFG/SHG light 121′, even a 2× selective output of the SHG/SFG light 121′ may provide output light 131′ that includes less SHG/SFG light 121′ than the visible light 111′. The selective outcoupling of the far-UVC light 121 as the output light 131 may be provided in one or more directions that differ from a direction of propagation of the pump light 111 in some embodiments.

While described in specific embodiments herein with reference to lasers that emit light 111′ at 440 nm and output couplers that output light 121′ at 220 nm, it will be understood that such specific emission wavelengths are mentioned by way of example only, and that any of the embodiments described herein (and/or components thereof) may be configured to emit or operate using other emission wavelengths such that the overall light output includes light in the far-UVC wavelength range. Likewise, while described primarily below with reference to example implementations of the nonlinear optical element 120 for second harmonic generation (referred to as an SHG element), it will be understood that, in any of the embodiments described and illustrated herein, the SHG element may be replaced by any nonlinear optical element 120 that is configured to generate light by sum frequency generation (referred to as an SFG element).

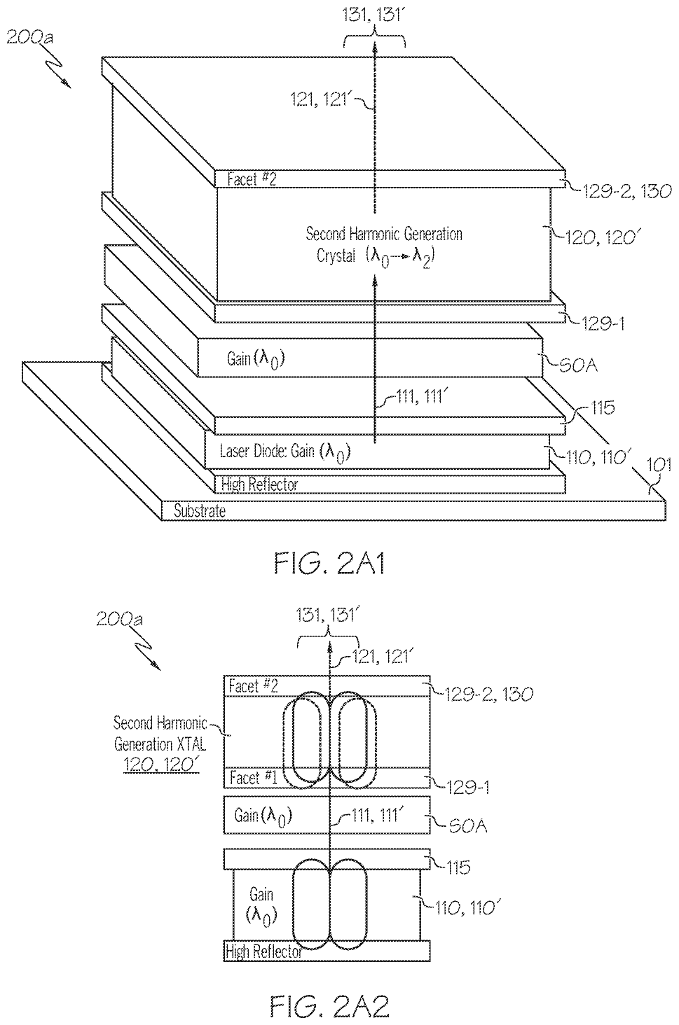

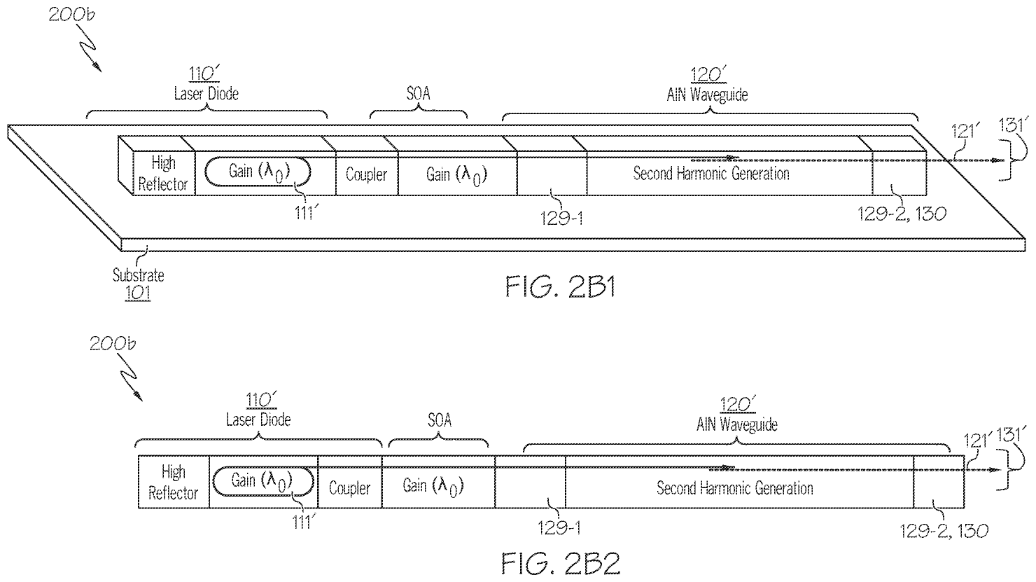

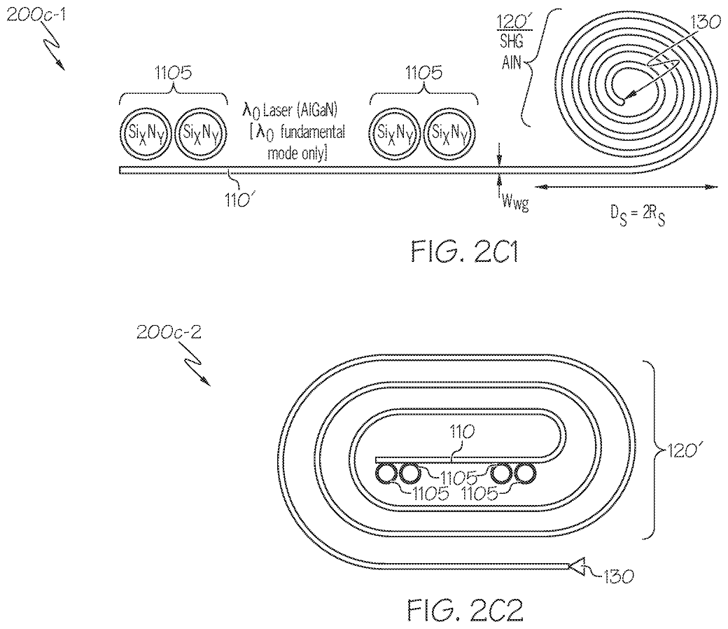

FIGS. 2A1 and 2A2 are schematic perspective and side views, respectively, illustrating elements of a UV light source 200a in a vertical linear arrangement (e.g., a Vertical External Cavity Surface Emitting Laser (VECSEL)) according to some embodiments of the present disclosure. FIGS. 2B1 and 2B2 are schematic perspective and top views, respectively, illustrating elements of a UV light source 200b in a horizontal linear arrangement according to some embodiments of the present disclosure.

The linear UV light sources 200a, 200b (in both the vertical arrangement shown in FIGS. 2A1 and 2A2 and the horizontal arrangement shown in FIGS. 2B1 and 2B2) include the light emitting element 110 and the nonlinear optical element 120 integrated in a monolithic structure 190. In particular, the linear UV light sources includes a light emitting element 110 (e.g., a laser diode 110′ including a gain material configured to generate light output in the blue part of the visible spectrum with a wavelength of about 440 nm), an input coupling element 115, an optional semiconductor optical amplifier (SOA) with gain at about 440 nm, a monolithically integrated nonlinear optical element 120 (e.g., an AlN waveguide with nonlinear optical properties configured for Second Harmonic Generation of frequency doubled light in the far UV part of the visible spectrum near 220 nm), and two facets 129-1, 129-2. The first facet 129-1 has high reflectivity for both approximately 440 and approximately 220 nm light, and the second facet 129-2 has higher reflectivity for 440 nm light and lower reflectivity for 220 nm light to provide selective outcoupling of the far-UVC light 121′.

FIGS. 2C1 and 2C2 are schematic top views illustrating elements of UV light sources in a spiral arrangement according to some embodiments of the present disclosure. The spiral UV light sources 200c-1, 200c-2 may integrate the nonlinear optical element 120 (and in some embodiments, the output coupling element 130) in a spiral-shaped waveguide 220, which may be adjacent (in FIG. 2C1) or extend around (in FIG. 2C2) the light emitting element 110 (e.g., a laser diode 110′ including optical resonators 1105 configured to generate visible light 111′ with a wavelength of about 440 nm). The embodiments of FIGS. 2C1 and 2C2 may allow for increasing optical length substantially while maintaining a relatively small footprint. While bending of the waveguide may increase radiative losses, this may be advantageous in some embodiments, particularly for embodiments in which the far-UVC light 121′ can be continuously or quasi-continuously outcoupled (also referred to herein as distributed emission) along a length of the output element, which may avoid or mitigate phase matching requirements.

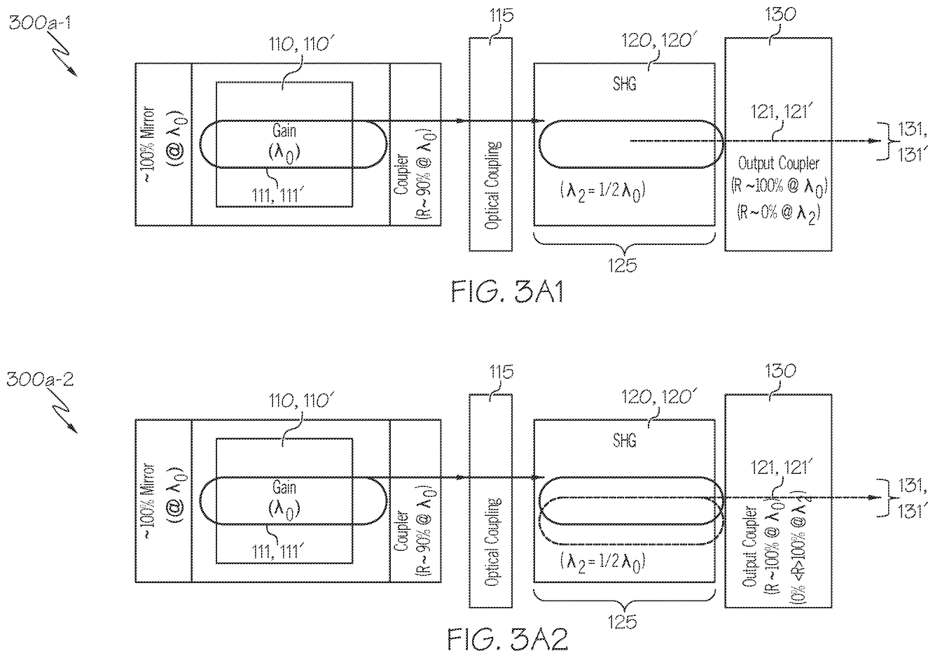

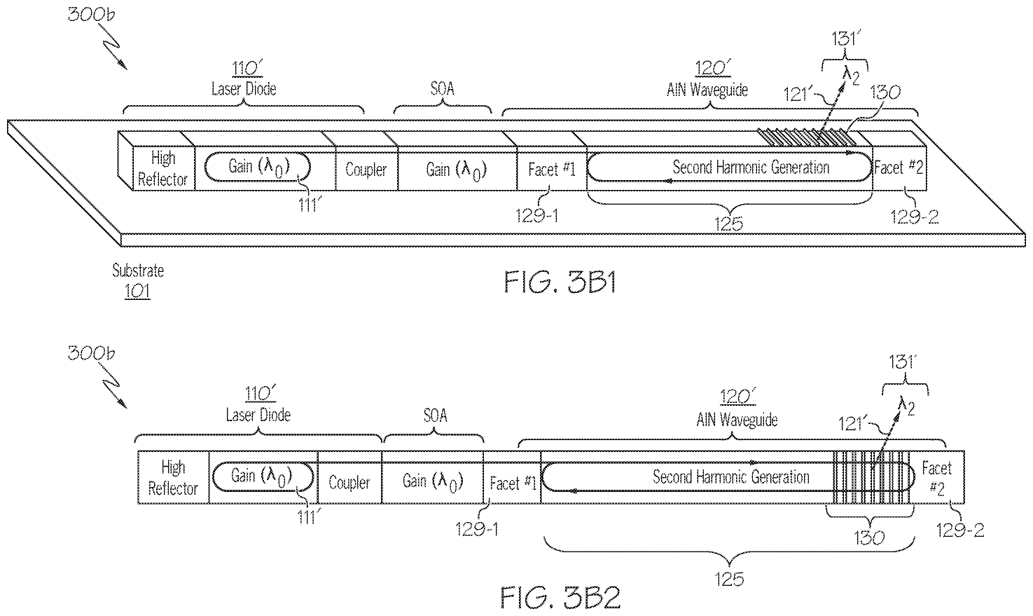

FIGS. 3A1 and 3A2 are schematic block diagrams illustrating elements of UV light sources 300a-1, 300a-2 including optical cavity enhancement according to some embodiments of the present disclosure. In the examples of FIGS. 3A1 and 3A2, the output coupling element 130 is configured to receive the far-UVC light 121′ from an optical cavity 125 that is at least partially resonant at the first frequency. For example, the optical cavity 125 may be optically resonant at the first (fundamental) wavelength/frequency of the visible light 111′, the second (e.g., harmonic) wavelength/frequency of the far-UVC light 121′, or both. The optical cavity 125 may include or may surround the nonlinear optical element 120 (e.g., separate from the lasing cavity 105 of the laser 110′) in some embodiments. By providing the non-linear crystal inside an optical cavity 125, the efficiency of conversion can be increased by providing for many passes of the pump light 111 through the nonlinear crystal. In this way the optical cavity 125 acts to optically “lengthen” the nonlinear optical element 120 (with respect to distance of light propagation) beyond the physical dimensions of the nonlinear optical element 120. Alternatively, cavity enhancement can be considered as recycling the pump light 111 over multiple passes. Furthermore, the optical cavity 125 may greatly increase the pump light field intensity in accordance with the quality (Q) factor of the cavity 125. Because the efficiency of nonlinear wavelength conversion (e.g., SFG/SHG) may depend monotonically on pump field intensity, the use of an optical cavity 125 can improve SFG/SHG efficiency for each of the passes.

In the example of FIG. 3A1, the nonlinear optical element 120 (or the optical cavity 125 in which the nonlinear optical element 120 is provided) is shown as resonant at the pump wavelength, and is non-resonant (or has high loss, i.e., emission) at the SHG/SFG (e.g., far-UV) wavelengths, also referred to herein as a singly resonant configuration. In a singly resonant configuration, the pump wavelength is resonant such that its intensity will “build” (i.e., increase) in the optical cavity 125, while the SHG/SFG wavelength will not build up substantially because the optical cavity 125 is not resonant at the SHG/SFG wavelength. While not wishing to be bound by theory, in such a singly resonant configuration, requirements for phase matching may be diminished or relaxed. Phase matching may be sufficient to build the intensity of the SHG/SFG wavelength over a single pass over the length of the nonlinear optical element 120.

In the example of FIG. 3A2, the nonlinear optical element 120 (or the optical cavity 125 in which the nonlinear optical element 120 is provided) is shown as resonant at both the pump wavelength, and at the SHG/SFG (e.g., far-UV) wavelengths, also referred to herein as a doubly resonant configuration. If both the pump and the SHG/SFG wavelengths are resonant with the nonlinear optical element 120, then both pump and SHG/SFG intensities may build up in the optical cavity 125, and phase matching requirements may be stricter. Indeed, doubly resonant operation may not be possible without realizing phase matching between the two wavelengths. Between each pass, the SHG/SFG light 121′ that is generated may be (partially or totally) coupled out of the optical cavity 125. The optical cavity 125 may be configured to couple in the maximum amount of pump laser light 111, while preventing the pump light 111 from leaking out on each pass.

More generally, embodiments of the present disclosure include implementations that are singly or doubly resonant. Although the figures may show gaps between elements (e.g., in FIGS. 1B, 3A1, 3A2), it will be understood that the illustration of such gaps may merely be provided to distinguish functional components from one another, and does not imply that the light necessarily propagates through free space between components or elements. For example, as noted above, embodiments with monolithic integration of components may have no free-space light propagation between components, while embodiments with hybrid integration may involve some free-space light propagation.

FIGS. 3B1 and 3B2 are schematic perspective and top views, respectively, illustrating elements of a UV light source 300b including optical cavity enhancement according to some embodiments of the present disclosure. FIGS. 3B1 and 3B2 illustrate an implementation of the elements shown in FIG. 3A1 on a single chip, in a linear, horizontal geometry.

In FIGS. 3B1 and 3B2, the nonlinear optical element 120 is implemented as an AlN waveguide, and is provided in an optical cavity 125 indicated by Facet #1 129-1 and Facet #2 129-2. This optical cavity 125 recycles the pump light 111 in order to enhance the nonlinear effect of SHG/SFG, and is configured to avoid feeding pump light 111 back to the original pump laser diode 110′ in too high of a quantity. The output coupling element 130 is implemented as a grating on a portion (e.g., some or all) of the nonlinear optical element 120. The grating is of a diffraction order that is configured to selectively outcouple the SHG/SFG (e.g., far-UV) light out with an efficiency that may be optimized for overall performance. High levels of outcoupling of the far-UVC light 121′ would provide a singly resonant cavity (resonant only at the pump wavelength), while low levels of output coupling of the far-UVC light 121′ would provide in a doubly resonant cavity (i.e., resonant at both pump and SHG/SFG wavelength). As noted above, doubly resonant designs may have stricter requirements on phase matching and component design.

The grating shown in FIGS. 3B1 and 3B2 is one of many possible implementations of an output coupling element 130 that is configured to selectively couple the SHG/SFG light 121′ out of the nonlinear optical element 120. For example, the output coupling element 130 may be or may include at least one of a facet having a refractive index that is configured to selectively outcouple the far-UVC light 121′ in a first direction corresponding to a direction of propagation thereof, or a grating having a diffraction order that is configured to selectively outcouple the far-UVC light 121′ in a second direction, different than the first direction. The second direction may be orthogonal to the first direction of propagation of the visible light 111′ from the light emitting element 110. For example, the grating or facet may be configured to outcouple the far-UVC light 121′ in a direction that is normal to a surface of a substrate 101 (native or non-native) having the UV light source thereon.

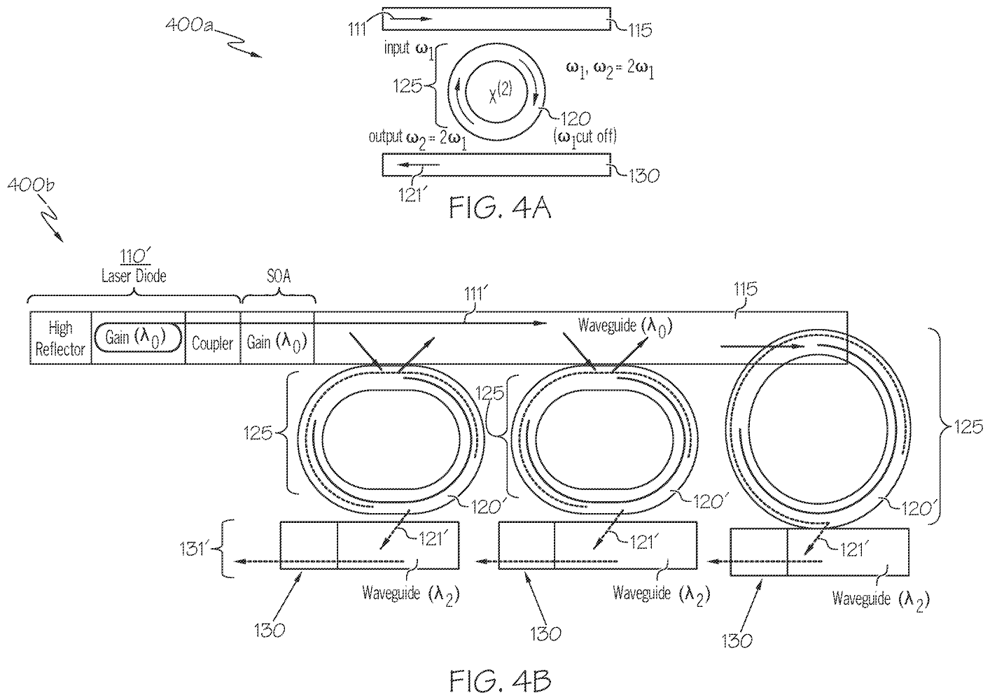

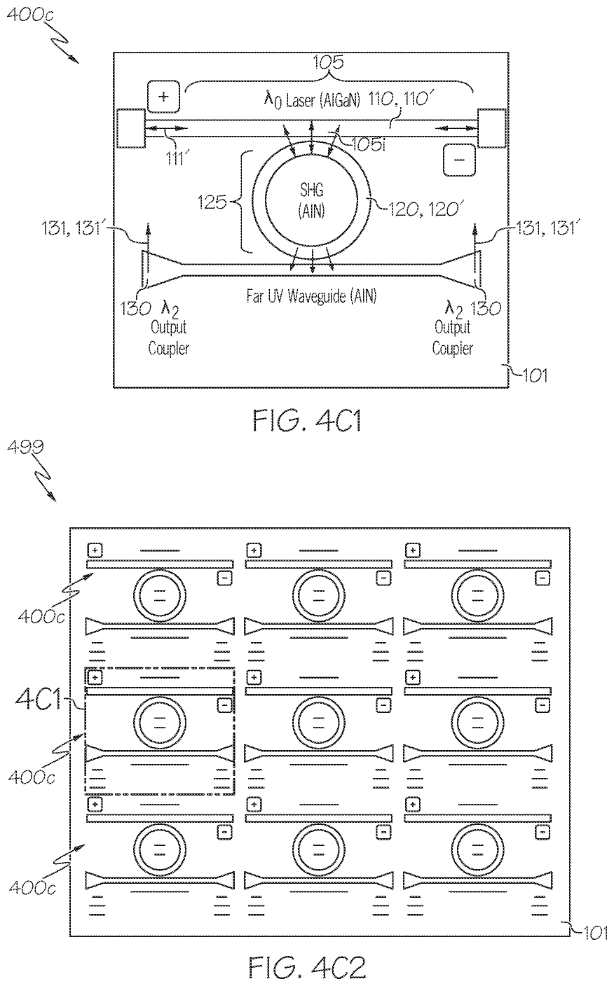

FIG. 4A is a schematic block diagram illustrating elements of a UV light source 400a including a nonlinear optical element 120 in a ring cavity configuration according to some embodiments of the present disclosure. This is another variation of a design in which the nonlinear optical element 120 employs resonant effects, using ring shaped optical cavities instead of linear cavities, to enhance SHG/SFG, shown in plan view.

As shown in FIG. 4A, the nonlinear optical element(s) 120 may be implemented in a ring configuration that defines the optical cavity 125, rather than being provided in an optical cavity 125. The ring cavity may be a circle, as shown for simplicity, or may be an oval, an ellipse, or other ring or closed shape. The nonlinear optical element 120 may be an AlN-based ring cavity in some embodiments. Pump light 111 of a first frequency ω₁ is coupled from the input coupling element 115 (shown as in input waveguide) partially or fully into the optical cavity 125 defined by the nonlinear optical element 120. The SHG/SFG light 121′ is coupled out of the nonlinear optical element 120 (selectively) into the output coupling element 130 (shown as including an output waveguide). The higher the coupling of SHG/SFG light 121′ out of the optical cavity 125, the lower the quality factor (Q) of the cavity and thus the closer the cavity is to being singly resonant. More generally, the optical cavity 125 may be at least partially resonant at both the pump and the SHG/SFG wavelengths.

In some embodiments, multiple nonlinear optical elements 120 (e.g., crystals or cavities) may be provided per pump laser 110′, each with a respective output coupling element 130. For example, as the input coupling element 115 may not transfer 100% of the pump light 111′ to the first SHG element, multiple different SHG/SFG elements 120′ or cavities may be arranged to receive light from a common input coupling element 115, and thus, to be pumped by a single pump laser 110′. That is, because the input coupling element 115 may be imperfect, any light that does not couple into the first SHG/SFG element 120′ or cavity may be provided to the next or subsequent SHG/SFG element 120′ or cavity. As such, each subsequent SHG/SFG element 120′ or cavity may receive “leftover” light that was not coupled into the previous SHG/SFG element 120′ or cavity. Further embodiments may use a photonic integrated circuit (PIC) to divide the pump light 111 prior to distribution across the various SHG/SFG elements 120′ or optical cavities 125.

FIG. 4B is a schematic top view illustrating elements of a UV light source 400b including a plurality of nonlinear optical elements 120 in ring cavity configurations that are sequentially arranged according to some embodiments of the present disclosure. As shown in FIG. 4B, the UV light source 400b includes an input coupling element 115 (shown as a waveguide) that is configured to receive the visible light 111′ from the light emitting element 110, with a plurality of nonlinear optical elements 120 sequentially arranged along the waveguide to receive the visible light 111′ from the light emitting element 110.

As noted above, coupling from the waveguide to a nonlinear optical element 120 may be less than 100% efficient. In fact, increasing Q of the ring cavity may demands that the coupling ratio be restrained from being too large. Understood differently, a larger coupling ratio may mean that a large fraction of the pump light 111 could leak from the ring back into the waveguide with every cycle around the ring cavity. As such, it may be advantageous to provide a plurality of nonlinear optical elements 120 along the length of a waveguide, each of which “taps” pump light 111 that was not coupled into the previous nonlinear optical element 120 in the arrangement sequence. In particular, FIG. 4B illustrates a Photonic Integrated Circuit (PIC) in which a light emitting element 110 (e.g., a blue (approximately 440 nm) single mode laser 110′) is integrated with a linear (e.g., AlN) waveguide as the input coupling element 115. The input coupling element 115 is coupled to one or more nonlinear optical elements 120 (e.g., AlN ring resonators) each having a respective optical cavity 125, which generate SHG/SFG light 121′ in the far-UVC (approximately 220 nm) wavelength range. The far-UVC light 121′ from each nonlinear optical element 120 is then coupled into a respective linear (e.g., AlN) waveguide, each of which includes either a low reflectivity (with respect to the far-UVC light 121′) exit facet or a grating as an output coupling element 130.

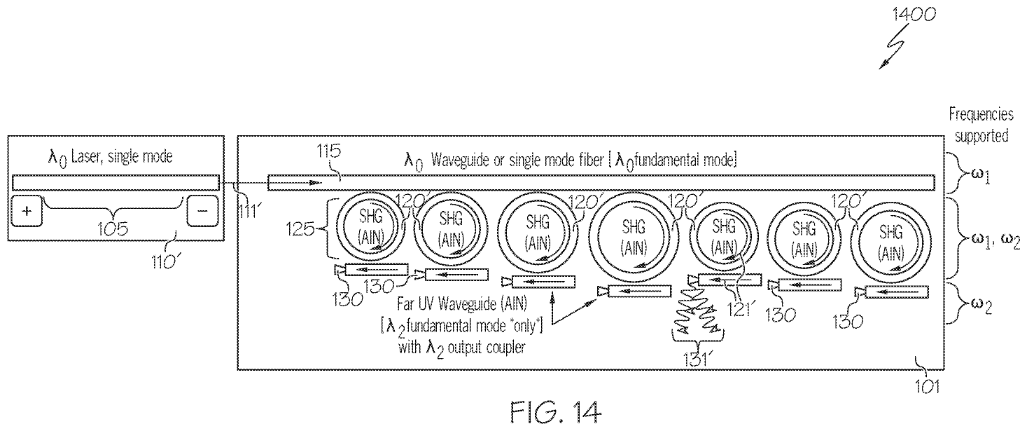

FIG. 4B thus illustrates (i) branching or splitting of the pump light 111 into a plurality of SHG/SFG elements 120′, and (ii) configuring the design of each subsequent SHG/SFG element 120′ to improve or optimize overall device performance (as also shown in further detail in FIG. 14). As also shown in FIG. 4B, the nonlinear optical elements 120 may not necessarily be identical to one another. For example, some embodiments may provide different coupling ratios for subsequent SHG/SFG ring cavities, as indicated by the final SHG/SFG ring (on the far right side of FIG. 4B) having different geometry. More generally, respective ones of the nonlinear optical elements 120 may have different dimensions and/or even different materials, and the output coupling element 130 may include a plurality of output coupling elements 130 that are respectively configured to selectively outcouple the far-UVC light 121′ as output light 131′ from the respective ones of the nonlinear optical elements 120.

FIG. 4C1 is a schematic top view illustrating elements of a UV light source 400c including a nonlinear optical element 120 coupled to an intra-cavity portion 105i of the light emitting element 110 according to some embodiments of the present disclosure. FIG. 4C2 is a schematic top view illustrating an array 499 of UV light sources 400c that respectively include a nonlinear optical element 120 coupled to an intra-cavity portion 105i of a light emitting element 110 according to some embodiments of the present disclosure.

As shown in FIGS. 4C1 and 4C2, a (ring) cavity resonant SHG/SFG element 120′ is coupled to a pump laser 110′ on the same chip or substrate 101 (e.g., a native substrate) in FIG. 4C1, with multiple chips in an array 499 on a substrate 101 (e.g., a native or non-native substrate) in FIG. 4C2. Each nonlinear optical element 120 is arranged and configured to receive visible light 111′ from an intra-cavity portion 105i between first and second ends of a respective lasing cavity 105 (also referred to herein as an “intra-cavity-tap” configuration), in contrast to the configurations shown in previous embodiments where the nonlinear optical element 120 (s) are arranged to receive light output from an end of the lasing cavity 105 (also referred to herein as “external cavity-tap” configurations). That is, the terms intra-cavity coupling or tapping and external-cavity coupling or tapping may be used herein to differentiate between relative positions of the nonlinear optical elements 120 with respect to the light emitting element 110, for light coupling into the nonlinear optical elements 120.

As shown in FIG. 4C1, in the intra-cavity-tap configuration, the light output from the laser 110′ or other light emitting element 110 may only traverse one interface to be input to the optical cavity 125 of the nonlinear optical element 120, and thus, relatively high intensity intra-cavity light 111′ may be in-coupled to the nonlinear optical element 120. In the external cavity-tap configuration, the light output from the laser 110′ or other light emitting element 110 must pass through the end of the lasing cavity 105 (or other optical interface of the light emitting element 110), and then across a waveguide or other input coupling element 115 to be input to the optical cavity 125 of the nonlinear optical element 120. Because at least two optical interfaces between elements may be present in the external cavity-tap configuration (e.g., a waveguide having respective interfaces with the lasing cavity 105 and the nonlinear optical element 120), the light input to the nonlinear optical element 120 may be of lower intensity comparison to the intra-cavity-tap configuration. Also, in the external cavity-tap configuration, the light being tapped by the nonlinear optical element 120 propagates in a single direction (the direction of output from the laser 110′) relative to the nonlinear optical element 120. However, in the intra-cavity-tap configuration, the light propagates in two directions (between opposing ends of the lasing cavity 105), as indicated by the dual pointed arrows.

FIG. 4C3 is a graph illustrating vernier frequency selection for determining optical cavity 125 size (including height, width, and circumference/length) of a ring-shaped nonlinear optical element 120 according to some embodiments of the present disclosure. As shown in FIG. 4C3, the size of the optical cavity 125 may be tuned to correspond to a free spectral range (FSR) with resonances that (only) match specific modes of the pump laser 110′. By increasing the FSR beyond the spectral width of the gain of the laser, it may thus be possible for the nonlinear optical element 120 to modify the operation of the pump laser 110′, and thereby force more (or up to all) of its intra-cavity power to frequencies that are relevant to the SHG/SFG cavity, thereby increasing efficiency of the system. For example, providing an SHG/SFG ring 120′ at the edge of the lasing cavity 105 of an otherwise multimode laser 110′ may modify the laser 110′ into single mode operation.

While the intra-cavity-tap configuration is described herein with respect to far-UVC light 121′ generation where high SHG/SFG efficiency may be paramount, it may be used for light generation at other wavelengths. Also, it will be understood that, although the nonlinear optical element 120 is coupled to the interior of the lasing cavity 105 in FIGS. 4C1 and 4C2, the SHG/SFG optical cavity 125 (ring) is a distinct optical cavity 125 from the lasing cavity 105. In other words, the embodiments of FIGS. 4C1 and 4C2 illustrate two distinct or separate cavities per UV light source—the lasing cavity 105 of the pump laser 110′, and the optical cavity 125 of the nonlinear optical element 120.

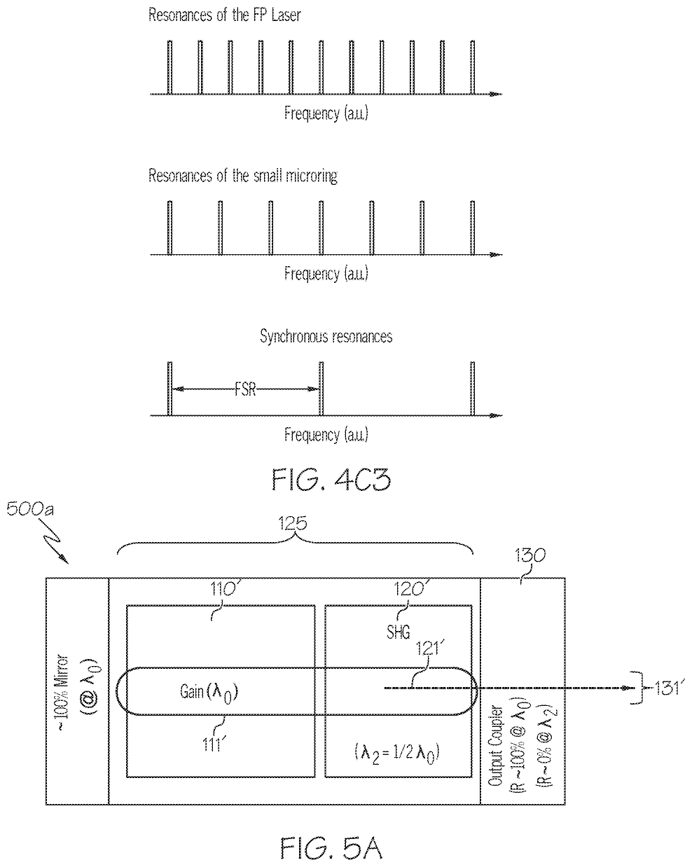

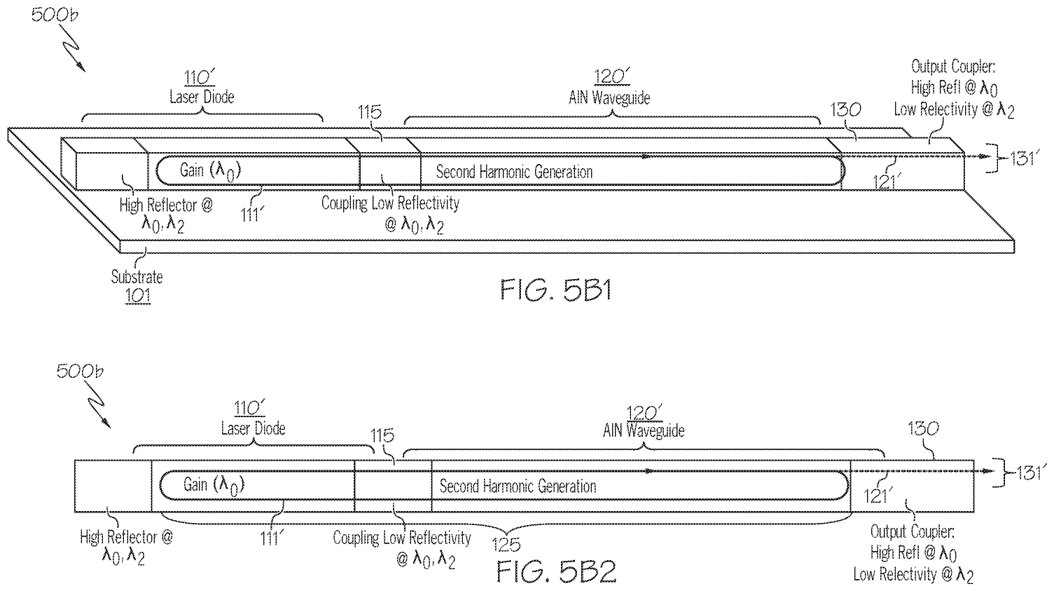

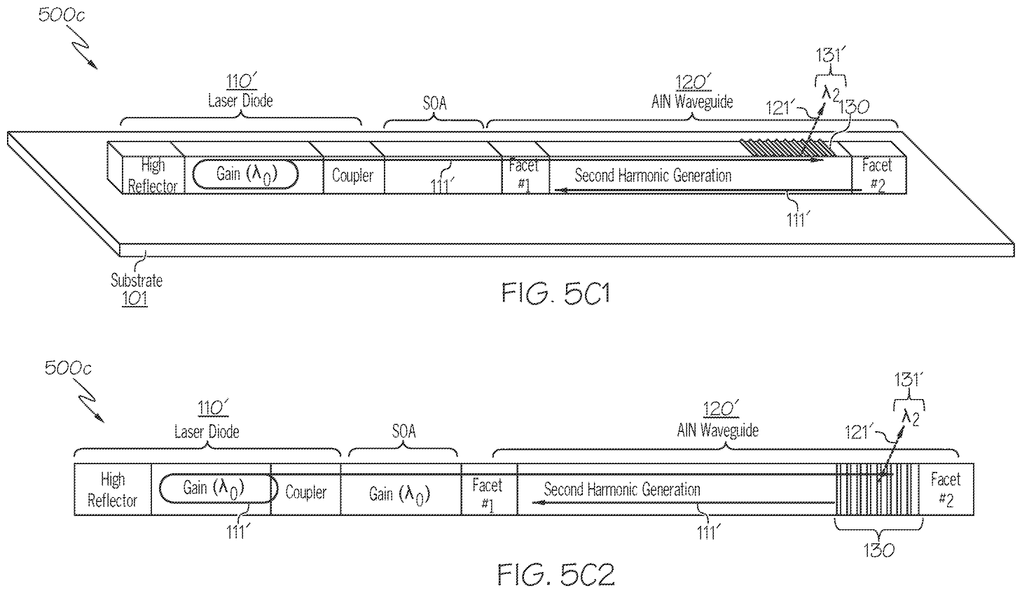

FIG. 5A is a schematic block diagram illustrating elements of a UV light source 500a in which the light emitting element 110 and the nonlinear optical element 120 are provided in a same or shared optical cavity 125 according to some embodiments of the present disclosure. That is, the nonlinear optical element 120 may be integrated into the same, single cavity as the gain material of the pump laser 110′, such that the cavity of the laser 110′ (i.e., the lasing cavity 105) or other light emitting element 110 is shared with the nonlinear optical element 120 that provides SHG/SFG. It will be understood that the shared optical cavity 125 configuration (also referred to herein as an intra-cavity-SHG/SFG configuration) shown in FIG. 5A is distinct from the “intra-cavity-tap” configuration where the nonlinear optical element 120 is coupled to the interior portion of the lasing cavity 105, but the optical cavity 125 of the nonlinear optical element 120 is distinct from the lasing cavity 105.

As shown in FIG. 5A, the shared optical cavity 125 may only be resonant at the pump wavelength, i.e., in a singly resonant configuration. There may be no need for gain at the SHG/SFG wavelength, as it may be impossible for the SHG/SFG wavelength to pass through the gain material (as the gain material is likely to be absorbing and not transparent at the SHG/SFG wavelength). That is, the shared optical cavity 125 need not be doubly resonant at both the first (pump) and second (SHG/SFG) frequencies, as the second harmonic frequency may be absorbed by the gain region and thus may not build up intensity by optical resonance. Instead, the shared optical cavity 125 may provide the SHG/SFG light 121′ to an output coupling element 130 that is configured to selectively outcouple the SHG/SFG photons as output light 131′ in a manner similar to that shown and described in other embodiments herein.

Advantages of this approach may include allowing the optical field of the fundamental (pump) wavelength to be far higher than that output from the light emitting element 110. The shared optical cavity 125 may be designed or otherwise configured with as high Q as possible (e.g., with a reflectivity of the output coupling element 130 of up to about 100%) at the pump wavelength in order to increase or maximize intracavity field strength. However, in some embodiments the Q may be reduced (e.g., the output coupling element 130 may have less than 100% reflectivity at the pump wavelength) in order to couple out some fraction of the pump wavelength for other purposes.

FIGS. 5B1 and 5B2 are schematic perspective and top views, respectively, illustrating elements of a UV light source 500b in a same or shared optical cavity 125 with an output coupling element 130 implemented as a reflective facet configured for selective light extraction according to some embodiments of the present disclosure. In particular, FIGS. 5B1 and 5B2 illustrate an implementation of the elements shown in FIG. 5A on a single chip, in a linear, horizontal geometry.

As shown in FIGS. 5B1 and 5B2, the UV light source 500b includes a light emitting element 110 implemented as a laser diode 110′ configured to generate light in the blue part of the visible spectrum near about 440 nm, a nonlinear optical element 120 (monolithically integrated with the light emitting element 110) implemented as an AlN waveguide 120′ with nonlinear optical properties configured for generation of frequency doubled light in the far-UVC part of the visible spectrum near about 220 nm, a first facet with high reflectivity for both approximately 440 nm and approximately 220 nm light, and an output coupling element 130 implemented as a second facet with higher reflectivity for 440 nm light and lower reflectivity for 220 nm light (to provide selective outcoupling of the far-UVC light 121′), in a horizontal linear arrangement. The first and second facets define the shared optical cavity 125, which is resonant with respect to the 440 nm light (i.e., singly resonant). An input coupling element 115 with low reflectivity for both approximately 440 nm and approximately 220 nm light is provided between the light emitting element 110 and the nonlinear optical element 120. Optionally, semiconductor optical amplifier (SOA) with gain at approximately 440 nm may amplify the output of the light emitting element 110 and provide the resulting light to the input of the nonlinear optical element 120 in some embodiments.

FIGS. 5C1 and 5C2 are schematic perspective and top views, respectively, illustrating elements of a UV light source 500c in a same or shared optical cavity 125 with an output coupling element 130 implemented as an optical grating configured for selective light extraction in a horizontal linear arrangement according to some embodiments of the present disclosure. The UV light source 500c may be similar to the UV light source 500b of FIGS. 5B1 and 5B2, but includes additional elements that may be used to implement a shared or an intra-cavity-SHG/SFG configuration on a single chip, in a linear, horizontal geometry.

As shown in FIGS. 5C1 and 5C2, the UV light source 500c includes a light emitting element 110 (e.g., a laser diode 110′ configured to generate light in the blue part of the visible spectrum near about 440 nm), a nonlinear optical element 120 (e.g., an AlN waveguide 120′ with nonlinear optical properties monolithically integrated with the light emitting element 110 and configured for generation of frequency doubled light in the far-UVC part of the visible spectrum near about 220 nm), and a (optional) semiconductor optical amplifier (with gain at approximately 440 nm) therebetween. A first facet (with high reflectivity for both approximately 440 nm and 220 nm light) and a second facet (with high reflectivity for at least the approximately 440 nm light, and in some embodiments for both the approximately 440 nm and 220 nm light) define the shared optical cavity 125, which is resonant with respect to at least at the 440 nm light (i.e., singly or doubly resonant). The output coupling element 130 is implemented as second (or other order) grating, which is configured to selectively outcouple the 220 nm light from the optical cavity 125, while the 440 nm light (i.e., the pump wavelength) is highly contained.

Further embodiments of the present disclosure may provide both the nonlinear optical element 120 and the light emitting element 110 (e.g., the laser gain medium) inside the same or shared optical cavity 125, with the optical cavity 125 having a ring or other closed curve shape (also referred to herein as a “racetrack” configuration, including non-rotationally symmetric closed loops of any shape), with light propagation in one or more directions (e.g., a single direction, or in opposite directions).

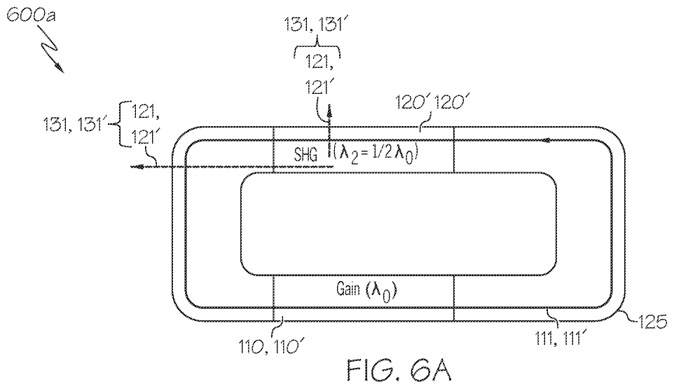

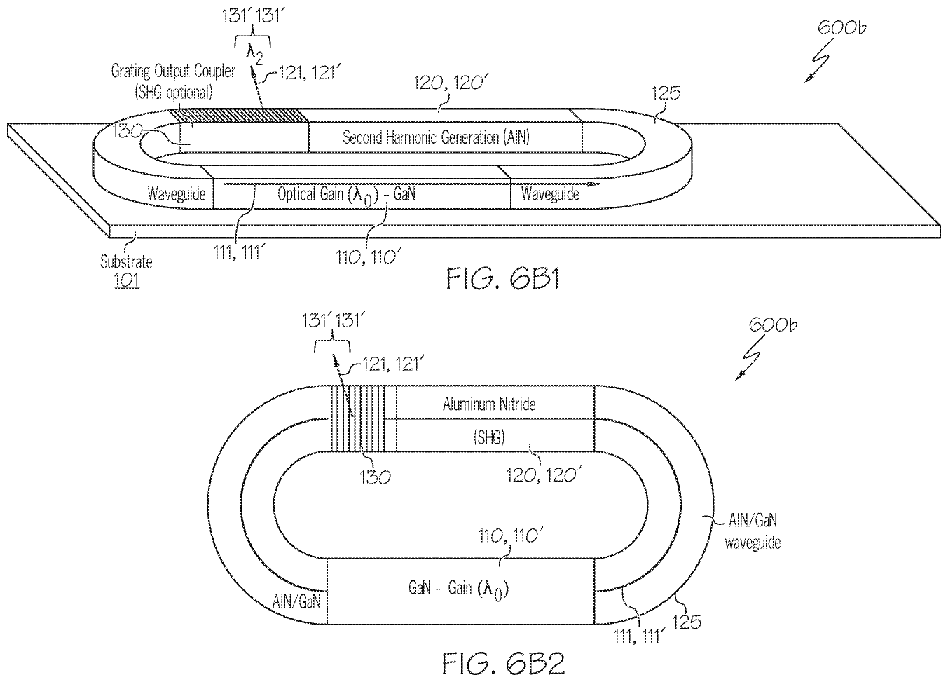

FIG. 6A is a schematic block diagram illustrating elements of a UV light source 600a in which the light emitting element 110 and the nonlinear optical element 120 are provided in a same or shared optical cavity 125 having a closed loop or racetrack (e.g., a rectangle with a semicircle at each end) configuration according to some embodiments of the present disclosure.

As shown in FIG. 6A, the optical cavity 125 has a closed curve shape that optically couples the laser gain medium and the SHG/SFG element 120′ therein, e.g., by curved waveguides with no reflective facets. The SHG/SFG wavelength may be selectively outcoupled either as it is generated (e.g., along a length of a portion of the optical cavity 125, also referred to herein as distributed emission), or after it is generated. This may be achieved, for example, by implementing the output coupling element 130 as a partially reflecting mirror or facet, a distributed Bragg reflector (DBR), or a second order diffraction grating to generate surface emission (e.g., in a direction orthogonal to the direction(s) of light propagation around the closed loop). The fundamental wavelength, meanwhile, can be confined completely within the optical cavity 125 with increased or maximum possible quality factor (Q).

FIGS. 6B1 and 6B2 are schematic perspective and top views, respectively, illustrating elements of a UV light source 600b in a same or shared optical cavity 125 having a closed loop or racetrack configuration with an output coupling element 130 implemented as an optical grating configured for selective light extraction according to some embodiments of the present disclosure. As shown in FIGS. 6B1 and 6B2, the UV light source 600b includes a light emitting element 110 (e.g., a GaN laser diode 110′ configured to generate light in the blue part of the visible spectrum near about 440 nm), and a nonlinear optical element 120 (e.g., an AlN waveguide with nonlinear optical properties monolithically integrated with the light emitting element 110 and configured for generation of frequency doubled light in the far-UVC part of the visible spectrum near about 220 nm), with curved waveguides (e.g., AlN/GaN waveguides) that optically couple respective ends of the light emitting element 110 and the nonlinear optical element 120, with no reflective facets therebetween. Avoiding or eliminating the use of reflective facets may allow for higher instantaneous pulse intensity, by avoiding limits associated with catastrophic mirror damage.

The output coupling element 130 is implemented as second (or other order) grating, which is configured to selectively outcouple the 220 nm light from the optical cavity 125. For example, the output coupling element 130 may include optical structures having a grating pitch that is configured based on the wavelength of the light to be outcoupled. The output coupling element 130 may be configured to direct the SHG/SFG wavelengths in a direction orthogonal to or otherwise out of a plane defined by the direction(s) of light propagation around the closed loop forming a surface emitting device), while the pump wavelength continues to propagate in the closed loop defined by the optical cavity 125. In some embodiments, the output coupling element 130 may also be a nonlinear optical element 120 (e.g., an AlN element with optical structures at a desired grating pitch), so as to selectively outcouple the far-UVC light 121′ as it is generated. In some embodiments, the optical cavity 125 may further include a section that is configured to form a saturable absorber 1305 that is configured to generate pulses of light of at the pump wavelength.

In particular, while some embodiments have been described by way of example with reference to continuous wave (CW) operation of the pump laser 110′, some embodiments may operate the pump laser 110′ in a pulsed mode, which can permit the operation of the devices at higher field intensities than CW. That is, the light emitting elements 110 in any of the embodiments described herein may be a laser diode 110′ that is configured to be driven in a continuous or pulsed manner.

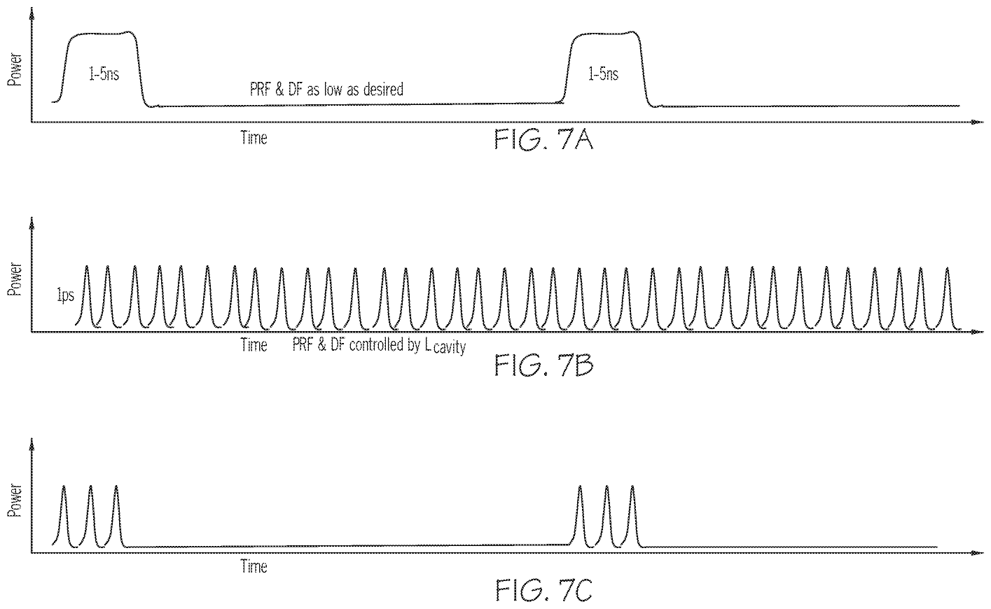

FIGS. 7A, 7B, and 7C illustrate example drive signals configured to provide pulsed light output from light emitting elements 110 in a UV light source according to some embodiments of the present disclosure. Referring to FIGS. 7A to 7C, SHG/SFG conversion may be higher when the (instantaneous) pump power is higher. Therefore the overall efficiency can be improved by maximizing the peak/average ratio of the pump laser 110′, in other words, by operating the pump laser 110′ to provide pulsed light output.

As shown in FIG. 7A, one method of obtaining pulsed light output is by direct modulation, that is, by providing a pulsed drive signal to the pump laser 110′, thereby activating and deactivating the laser diode 110′ to emit light for a desired pulse repetition frequency (PRF) and duration or duty factor (DF), e.g., with a pulse width of about 1 to 5 ns, with higher intensity or pulse output power than would be acceptable if operated continuously. That is, some embodiments may realize pulsed operation by use of an electrical drive circuit that provides short, high current pulses to a diode laser 110′. In this way, the laser 110′ may be “quasi CW” during the short period of high current application, but the low duty factor of the drive current may allow for higher transient operation powers.

As shown in FIG. 7B, another method of obtaining pulsed light output is to design or configure the laser diode 110′ to generate pulsed output even when driven continuously. One method to achieve this is by configuring the pump wavelength to be passively (or actively) mode locked, for example, by controlling the PRF and the DF of the laser diode 110′ based on the optical cavity 125 length Lcavity. FIG. 7B illustrates that a pulse “train” with pulse widths of as short as about 1 ps (or less) may achieved, with high pulse repetition frequency (e.g., c/Lcavity) to provide higher intensity or output pulse power than may be achieved by CW. For example, some embodiments may provide a saturable absorber 1305 inside the lasing cavity 105 to achieve passive mode-locking. Some embodiments may realize pulsed operation by active Q-switching.

As shown in FIG. 7C, the above or other pulsing strategies may be combined to realize even higher peak pulse powers and thus higher nonlinear conversion efficiency. In particular, FIG. 7C illustrates drive signals to provide a pulsed light output based on a combination of the methods shown in FIG. 7A (direct modulation) and FIG. 7B (continuous modulation), e.g., by using an electrical drive circuit to activate and deactivate the laser diode 110′ to emit light with a desired or predetermined PRF and DF, in combination with a saturable absorber 1305 in the lasing cavity 105 to provide the pulse widths as short as about 1 ps (or less). Also, while illustrated herein with reference to particular lasing cavities, it will be understood that other types of lasers may be used in any of the embodiments described herein. For example, a diffraction grating may be provided at ends of the laser gain medium to form a Distributed Feedback Laser (DFB) emitting in a single longitudinal mode. In some embodiments, a diffraction grating may be provided in a waveguide separate from the laser gain region forming a Distributed Bragg Reflector (DBR) laser emitting in a single longitudinal mode. More generally, embodiments of the present disclosure may use multiple operating methods (e.g., continuous wave and/or pulsed, by various methods or combinations thereof) and laser configurations (e.g., DFB, DBR) for the light emitting device, some of which may achieve higher SHG/SFG efficiency.

In order to monolithically integrate the light emitting element 110 with the nonlinear optical element 120, embodiments of the present disclosure may utilize various fabrication techniques to combine different materials. For example, some embodiments may utilize heterogeneous integration methods, such as microtransfer printing (MTP), to couple the laser 110′ and the nonlinear crystal if both are microscopic in size (e.g., with dimensions of about 0.5 μm to about 1000 μm). Microfabrication techniques may allow direct, end-to-end coupling of two optical components without the use of extra optical elements.

In some embodiments, as an alternative to micro assembly for the monolithic integration of two material sets into a single waveguide, epitaxial regrowth may be used on top of an existing waveguide that has been appropriately patterned. An example of such a concept is shown in FIGS. 2A1 and 2A2, where the gain material of the laser diode 110′ (e.g., GaN or other group III-nitride material) is formed, a section of the GaN is patterned and etched away, and an AlN layer or other nonlinear optical element 120 material is grown (e.g. by MOCVD, or MBE) such that a high quality optical interface is realized and the waveguide material changes without modifying the physical cross-section dimensions of the waveguide. In some embodiments, some or all elements of UV light sources described herein (e.g., the light emitting element 110, the nonlinear optical element 120, the output coupling element 130, optical cavities 125, and one or more waveguides therebetween) may be nitride-based materials.

Further embodiments may use MTP, pick and place, or other assembly techniques to arrange distinct active and passive optical elements on a non-native substrate, also referred to herein as hybrid integration. For example, respective light emitting elements 110 of one material may be formed and optically coupled to nonlinear optical elements 120 of a different material or material system on a non-native substrate, which is different from the source substrate of either the light emitting element 110 or the nonlinear optical element 120.

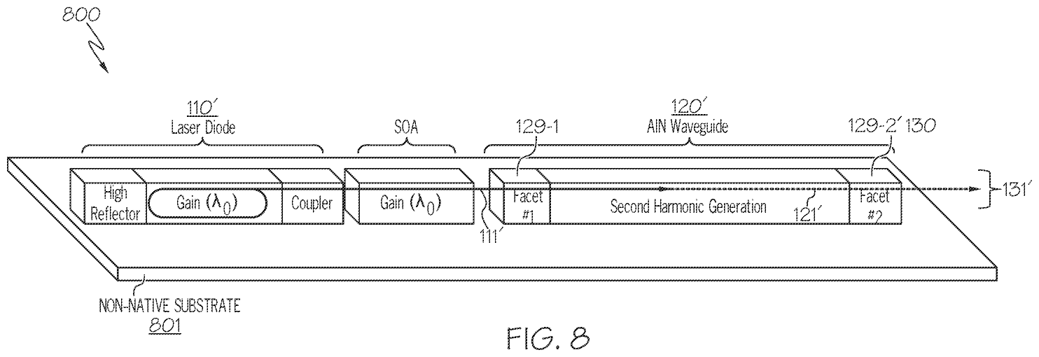

FIG. 8 is a schematic perspective view illustrating respective elements of a UV light source 800 arranged in a structure or configuration on a common non-native substrate 801 according to some embodiments of the present disclosure. As shown in FIG. 8, the UV light source 800 may be implemented as a Photonic Integrated Circuit (PIC) that includes respective elements as described herein as discrete components that are assembled onto a common substrate 801. In particular, the light emitting element 110 (e.g., a laser diode 110′ including a gain material configured to generate light output in the blue part of the visible spectrum with a wavelength of about 440 nm), the nonlinear optical element 120 (e.g., an AlN, BBO, or lithium niobate-based waveguide with nonlinear optical properties configured for Second Harmonic Generation of frequency doubled light in the far UV part of the visible spectrum near 220 nm), and the output coupling element 130 (e.g., a facet with higher reflectivity for 440 nm light and lower reflectivity for 220 nm light to provide selective outcoupling of the far-UVC light 121′) may be fabricated as respective elements and arranged on a non-native substrate 801. An optional semiconductor optical amplifier (SOA) with gain at about 440 nm may amplify the output of the light emitting element 110 and provide the resulting light to the input of the nonlinear optical element 120 in some embodiments.

In the “hybrid” integration example of FIG. 8, the optical coupling between the respective elements may be implemented by optical fiber or free-space propagation (with air interfaces therebetween). However, the UV light source 800 may be implemented by combinations of any of the fabrication techniques described herein, including (but not limited to) epitaxial regrowth, wafer bonding, microtransfer printing, pick and place, lithography, etc. It will be understood that, while illustrated herein with reference to monolithic integration of particular embodiments, any of these embodiments may be assembled using hybrid configurations as described herein. That is, any of the embodiments described herein may be assembled as unitary structures (with no air interfaces between components) or hybrid structures (with air interfaces between two or more components).

Similarly, it will be understood that embodiments of the present disclosure may include various types of optical cavities 125 and feedback structures that can provide the high quality factor Q for efficient operation. Examples of possible optical microcavities may include, but are not limited to a linear Fabry Perot cavity including of polished facet end mirrors, a linear Fabry Perot cavity including distributed (dielectric) Bragg reflector end mirrors, a linear optical Fabry Perot cavity including distributed feedback gratings, a linear optical cavity 125 including various photonic crystal designs, a ring cavity fabricated by a waveguide that closes on itself, and a ring cavity fabricated by a round or elliptical 2D or 3D disk structure.

Also, while embodiments herein have been primarily described with reference to optical second harmonic generation (SHG) from a pump laser 110′ (e.g., from blue light of about 400 nm to about 480 nm) to produce light emission of about 200 nm to about 240 nm, it will be understood that embodiments of the present disclosure may also include implementations in which higher order harmonic generation (e.g., third, fourth, and/or fifth order harmonic generation) is applied to the pump laser 110′ or other light 111 (including light of wavelengths appropriately higher than 400 nm to 480 nm) to produce output light 121 of 200 nm to 240 nm. That is, it will be understood that the second harmonic generation or frequency doubled light as described herein may be more generally be referred to as sum frequency (including harmonically multiplied) light generation, with the nonlinear optical element 120 implementing an optical frequency multiplier or other nonlinear frequency conversion device, in any of the embodiments described herein.

For some applications, more power in the far-UVC wavelength range may be desired than can be generated by a single UV light source. In such cases, multiple UV light sources (e.g., arranged in an array) may be provided on a common substrate 101 (native or non-native).

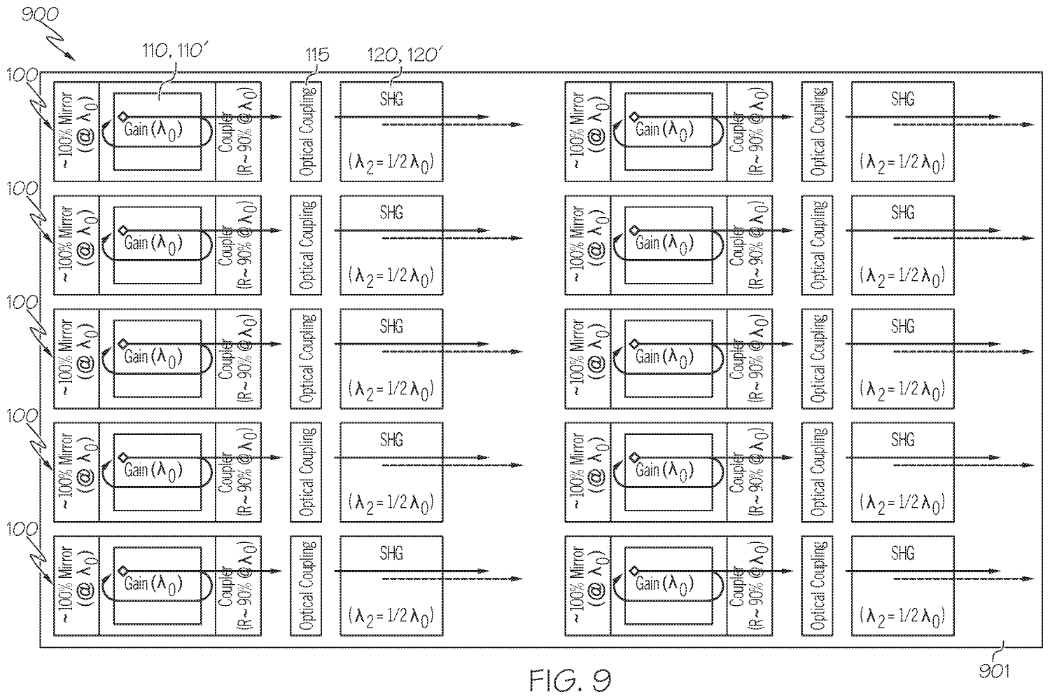

FIG. 9 is a schematic block diagram illustrating an array 900 of UV light sources 100 that respectively include a light emitting element 110 and a nonlinear optical element 120 according to some embodiments of the present disclosure. As shown in FIG. 9, an array 900 includes a plurality of the light emitting element 110 and the nonlinear optical element 120 on a common substrate 901. For example, some embodiments may utilize heterogeneous integration methods, such as microtransfer printing (MTP), to efficiently fabricate large arrays of similar or identical UV light sources 100 on a non-native substrate 901 in order to increase total optical power output from a single package. In other embodiments, rather than singulation and packaging of each UV light source separately, and subsequently aggregating the UV light sources on a common (i.e., non-native) substrate at the system level, arrays of identical (or non-identical) devices may be fabricated on the same chip (i.e., on the same native substrate) before packaging the chip. While illustrated as an array 900 of UV light sources 100 similar to that shown in FIG. 1B, it will be understood that any of the UV light sources described herein may be implemented in array form.

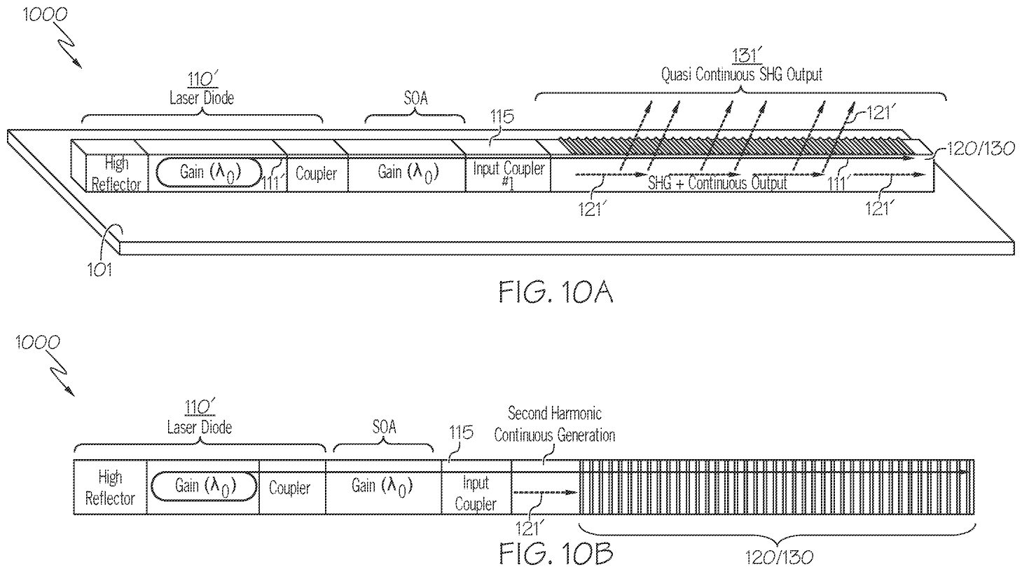

FIGS. 10A and 10B are schematic perspective and top views, respectively, illustrating elements of a UV light source 1000 including optical cavity enhancement with an output coupling element 130 configured to provide distributed emission and selective light extraction according to some embodiments of the present disclosure. As used herein, distributed emission may refer to a configuration of the nonlinear optical element 120 and the output coupling element 130 in which light (e.g., the far-UVC light 121′) is continuously or semi-continuously extracted as it is generated at multiple positions along the length of the component, rather than from one specific point or position. The distributed emission may or may not be collimated, and may or may not be coherent.

As shown in FIGS. 10A and 10B, the UV light source 1000 includes a light emitting element 110 (e.g., a laser diode 110′ configured to generate light in the blue part of the visible spectrum near about 440 nm), and nonlinear optical element 120 (e.g., an AlN waveguide with nonlinear optical properties monolithically integrated with the light emitting element 110 and configured for generation of frequency doubled light in the far-UVC part of the visible spectrum near about 220 nm). An optional SOA amplifies and provides the output of the light emitting element 110 to the input of the nonlinear optical element 120. In the embodiment of FIGS. 10A and 10B, the nonlinear optical element 120 and the output coupling element 130 are integrated into an output element 120/130 that is configured to outcouple the far-UVC light 121′ at a plurality of positions or continuously along a length thereof. In the example of FIGS. 10A and 10B, the output element 120/130 includes a waveguide material with alternating sections or regions, over which the SHG/SFG light 121′ is alternatingly generated (by the SFG/SHG section) and outcoupled (from the output coupling section). More generally, the output element 120/130 includes a plurality of nonlinear optical element 120 and output coupling element 130 regions (e.g., in a periodic or other alternating arrangement), which are configured to provide continuous or semi-continuous extraction or distributed emission of the SHG/SFG light 121′ along a length of the output element 120/130 (also referred to herein as a nonlinear optical output coupling element 120/130).

Output elements 120/130 with multiple integrated nonlinear optical and output coupling sections may be advantageous in that typical requirements or constraints with respect to phase matching between the SHG/SFG wavelength and the fundamental wavelength may be relaxed or may not be necessary. By semi-continuously extracting and recovering the SHG/SFG light 121′ from the output element 120/130, it may be possible to relax or eliminate constraints associated with phase matching between the SHG/SFG light 121′ and the pump light 111 inside the waveguide, as the SHG/SFG wavelength is not expected to co-propagate with the fundamental wavelength. That is, the SHG/SFG field intensity is not expected to accumulate within the waveguide; rather, the SHG light that is generated is outcoupled (in some fraction) to the outside world as it is generated. The output light 131 may thus primarily include the SHG/SFG light 121′, and in some instances may be substantially free of the pump light 111.

As such, the UV light source 1000 is free of phase matching (i.e., is not configured to match a first phase of the visible light 111′ with a second phase of the far-UVC light 121′). It will be understood that, although shown in the examples of FIGS. 10A and 10B with reference to a UV light source 1000 in a horizontal linear arrangement, output elements 120/130 including integrated nonlinear optical and output coupling elements 130 (e.g., arranged in an alternating manner along a length thereof) can be used in one or more other embodiments described herein, for example, embodiments including closed curve/racetrack optical cavity 125 configurations, spiral-shaped waveguides, or other output coupling element 130 configurations.

Also, while the example of FIGS. 10A and 10B illustrates the output element 120/130 as including second order gratings that selectively couple the SHG/SFG light 121′ out of the waveguide while confining the pump light 111 within the waveguide, embodiments of the present disclosure may include other types of output coupling elements 130 having wavelength-dependent optical characteristics configured for distributed emission or (semi-) continuous extraction of the SHG/SFG light 121′. Some further output element 120/130 configurations for selective extraction of the SHG/SFG light 121′ include, but are not limited to, dielectric material interfaces or stacks configured for wavelength dependent transmission, curves or tapers in waveguide geometry to obtain wavelength dependent effects.

For example, in some embodiments, the output element 120/130 may be configured to provide periodic poling of the SHG/SFG material (e.g. AlN) to accomplish “quasi” phase matching. In particular, the output element 120/130 may include alternating regions of AlN, each with different heights (relative to a substrate 101) and surface roughness. To address poor performance as a waveguide at λ₀, a capping layer that is index matched at λ₀ may be provided on top of the alternating AlN regions. That is, the output element 120/130 may include a plurality of periodically poled nonlinear optical sections of a first material, and an index-matched capping layer of a second material that is different than that of the nonlinear optical sections. Phase matching at the SHG/SFG frequency (e.g., λ₀/2) may not be needed, as the SHG/SFG light 121′ may be scattered out of the output element 120/130 with this configuration (such that the need for phase matching may be relaxed or obviated).

More generally, output elements 120/130 configured to provide distributed emission as described herein may include any optical structures (or combinations thereof) that are configured to confine the light of the fundamental wavelength (λ₀) output from the light emitting element 110 and radiate or outcouple the light of the SFG/SHG wavelengths (e.g., λ₀/2). As one example, in some embodiments, the output element 120/130 may be a waveguide that includes nanopores or defects therein, which have dimensions (or bandgaps) configured to affect only the SFG/SHG wavelengths (e.g., λ₀/2) while leaving the fundamental wavelength substantially unaffected. That is, the output coupling element 130 may be implemented as a waveguide that includes nanopores or defects having dimensions configured to be index-mismatched at the second (SHG/SFG) frequency of light, but to not substantially affect propagation of the first (fundamental) frequency of light.

For example, in some embodiments, the output element 120/130 may be a waveguide that includes or incorporates two (or more) different materials, having respective optical indexes that are matched at the fundamental wavelength λ₀, but are mismatched at the SHG/SFG wavelengths (e.g., λ₀/2), and roughened (e.g., at an interface between the materials) so that the light of the SHG/SFG wavelengths is scattered out of the output element 120/130, while the light of the fundamental wavelength is confined therein (also referred to herein as a confined mode).

Conversely, in some embodiments, the output element 120/130 may be a waveguide that includes or incorporates two (or more) different materials, whereby the two materials are index matched at the SHG/SFG wavelengths (e.g., λ₀/2) but mismatched at the fundamental wavelength λ₀, such that the first material provides a confined mode for the fundamental wavelength λ₀ while the SHG/SFG wavelengths can occupy modes in the second (optically “thicker”) material. Roughening or other scattering structures may be provided at a top of the second material (e.g., opposite an interface with the first material) such that the SHG/SFG wavelengths (e.g., λ₀/2) are preferentially scattered out of the waveguide, while the fundamental wavelength λ₀ remains confined in the higher index material. That is, the output coupling element 130 may be implemented as a waveguide including first and second materials having relative dispersion curves configured such that first and second optical indexes thereof are matched at one of the first (fundamental) and second (SHG/SFG) frequencies, but mismatched at the other.

As yet another example, in some embodiments, rather than combining the nonlinear optical element 120 with the scattering or output coupling element 130 over the length of the output element 120/130, the output element 120/130 may be a waveguide that includes distinct or separate nonlinear optical element 120 and output coupling element 130 sections. The SHG/SFG sections 120′ may be relatively short (along the direction of propagation of the fundamental wavelength light 111) so that phase matching may not be required or necessary over the length of the optical element. A relatively long output coupling section 130 is provided after (relative to direction of propagation of the fundamental wavelength light 111) each SHG/SFG section 120′, and may include different first and second materials that are configured to scatter or outcouple the light of the SHG/SFG wavelengths (e.g., λ₀/2) out of the waveguide while confining the light of the fundamental wavelength (λ₀). The sequence of alternating SHG/SFG sections 120′ and output coupling sections 130 may be repeated (e.g., periodically) along the direction of propagation of the fundamental wavelength light 111 in order increase SHG/SFG. Respective materials for the SHG/SFG sections 120′ and output coupling sections 130 may be selected such that the optical index at the fundamental wavelength (λ₀) is matched and confined across all periods of the structure. That is, at the SHG/SFG wavelengths (e.g., λ₀/2), the output element 120/130 may alternate between SHG/SFG sections 120′ and output coupling sections 130 to extract the SHG/SFG wavelengths of light, while at the fundamental wavelength (λ₀), the output element 120/130 may be continuous and may confine the light of the fundamental wavelength at all locations along the propagation direction.

Some photonic Integrated Circuits (PIC) as described herein may be based upon the GaInAlN material system, may be scalable to high volumes, and can leverage the extensive growth and fabrication infrastructure that has been deployed for the manufacture of white LEDs. The PICs described herein may be configured to emit an engineered monochromatic output at one or more wavelengths of choice between 200-240 nm, which in some embodiments can eliminate the use of or need for an optical filter, which may provide significant cost savings. The light emitting element 110 may be a laser 110′ that emits light in the 400-480 nm (blue/violet) wavelength range, and the nonlinear optical element 120 (which may be implemented as an engineered waveguide) may sum or double the frequency of the light input from the laser 110′ based on SFG or SHG, such that far-UVC light 121′ is generated at the desired wavelength. The light is then coupled out of the chip, in an out of plane direction (e.g., substantially normal to its surface plane) similar to the emission of a Vertical Cavity Surface Emitting Laser (VCSEL). Furthermore, the power output can be increased beyond what one device is capable of, simply by designing the PIC with a monolithic array of devices on a single chip analogous to a VCSEL array.

Referring again to FIGS. 4C1 to 4C3, an example PIC architecture according to some embodiments of the present disclosure includes a light emitting element 110 implemented as a linear single frequency pump laser diode 110′ (in this example, an AlGaN laser diode) coupled to a resonator nonlinear optical element 120 (which, in the example of FIG. 4C1, is ring-shaped and formed from AlN), which is coupled to an output coupling element 130 implemented as a waveguide (in this example, AlN) for extracting the far-UVC light 121′. Ring-shaped nonlinear optical elements 120 may also be referred to herein as ring resonators. However, it will be understood that the nonlinear optical elements 120 need not be ring-shaped, and other nonlinear optical element 120 designs may be used in embodiments described herein.

As shown in FIG. 4C2, each emitter of an emitter array 499 includes a laser 110′ (e.g., configured to emit 440 nm light; which may more generally be referred to herein as input light) that builds high internal optical intensity and couples a fraction of its light into the neighboring nonlinear optical element 120 (AlN ring resonator), which is resonant at this mode. The coupling (shown by arrows in FIG. 4C1, indicating directions of laser light 111 propagation) may be in-plane (i.e., along the plan view or x-y axis in the figures) or vertical (i.e., perpendicular to or out of the page), and in some embodiments, across a gap (e.g., on the order of microns) between the laser 110′ and the nonlinear optical element 120. The positive symbol represents the electrical anode and the negative symbol represents the electrical cathode of the laser 110′. Respective mirrors or other low-loss reflective elements may be provided at opposite ends of the laser 110′.

In particular embodiments, the laser 110′ may be implemented with an integrated waveguide or other integrated lasing cavity 105 (shown as bidirectional) in some embodiments. The pump (e.g., 440 nm) light that is coupled into the AlN nonlinear optical element 120′ (ring resonator) generates far-UVC (e.g., 220 nm) light because of the nonlinear response of AlN. This second harmonic generation (SHG) or sum frequency generation (SFG) process builds far-UVC (e.g., 220 nm) light more efficiently because of the high Q of the cavity at the frequency of the pump light. The far-UVC (e.g., 220 nm) light is coupled (selectively) out of the nonlinear optical element 120′ (ring resonator) and into a neighboring output waveguide, likewise across a gap (e.g., on the order of microns) therebetween in some embodiments. The output waveguide may be formed of, for example, AlN, SiO₂, or other materials, and may or may not be linear in some embodiments. For example, a ring resonator 120′ may have separate nonlinear and (selective) output coupling sections in some embodiments. The far-UVC light (or, more generally, SHG/SFG light 121′) may be output by respective output coupling elements 130 at opposing ends of the output waveguide, as output light 131′ propagating in a direction perpendicular to or otherwise out of the page, to provide surface emission (similar to a VCSEL). The output waveguide may be narrower than the lasing cavity 105 (e.g., may be less than 100 nm in width) in some embodiments.

It should be noted that 440 nm light can couple in two directions: from the laser 110′ into the ring resonator (forward coupling) and in reverse (reverse coupling). The reverse coupling can provide benefits: by coupling the two optical cavities 125 (the lasing cavity 105 and the SHG/SFG cavity), it can be ensured that the single longitudinal lasing mode of the laser 110′ is the correct mode needed for pumping the SHG process. This can ensure that the only frequency that is supported is matched to both resonators, as illustrated by the graphs shown in FIG. 4C3. The top graph illustrates resonances of the laser 110′ (including 440 nm light); the middle graph illustrates resonances of the ring resonator (including the 440 nm light and 220 nm higher order light resulting from the second harmonic generation or frequency doubling); and the bottom graph illustrates the synchronous resonances of the far UV output waveguide (including the 220 nm light). The physical sizes of the devices shown in herein are not to scale. The cross sectional dimensions of the respective elements may be configured to provide the desired wavelength, mode requirements, phase matching, etc.

Further embodiments are described below with reference to various configurations of a single emitter or UV light source. It will be understood that, as noted above, any of the configurations described herein may be implemented in an array including a plurality of emitters, which may or may not be of the same or identical emitter configuration. That is, any of the UV light source configurations may be combined herein in any way.

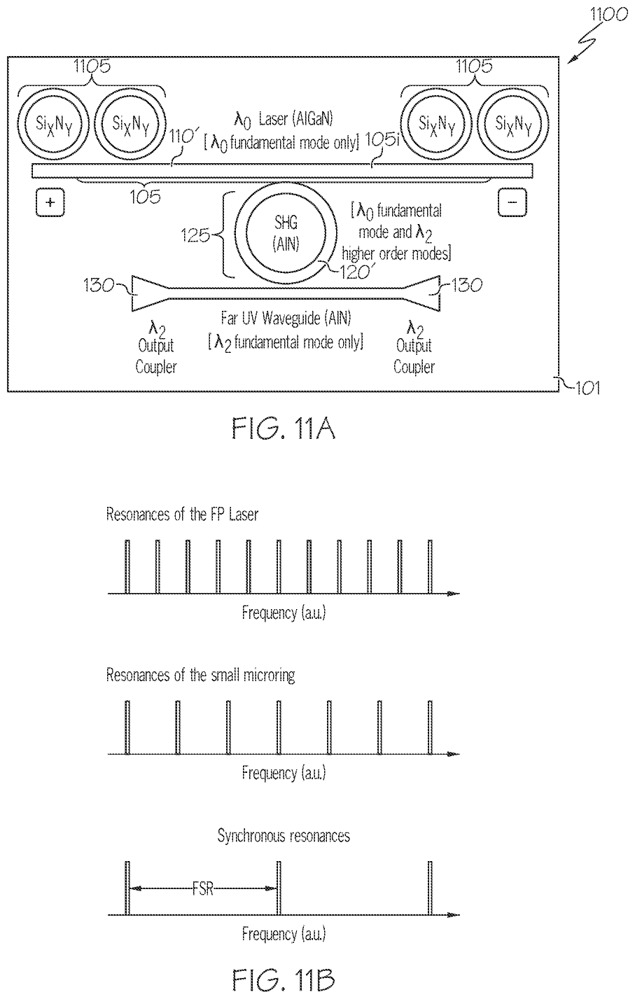

FIG. 11 is a schematic top view illustrating an example combination of various elements of a UV light source 1100 including a nonlinear optical element 120 coupled to an intra-cavity portion 105i of a light emitting element 110, in combination with ring resonators at respective ends 105a, 105b of the light emitting element 110 and an output coupling element 130 configured for selective light extraction according to some embodiments of the present disclosure. In the example of FIG. 11, the light emitting element 110 is implemented as a laser diode 110′, with one or more optical resonators 1105 that at opposing first and second ends of the lasing cavity 105 (shown as including an AlGaN waveguide).

As shown in FIG. 11, the nonlinear optical element 120 is implemented as a SHG/SFG element 120′ having a ring-shaped optical cavity 125, and is intra-cavity-tapped to the lasing cavity 105 of the laser diode 110′ as a pump laser 110′. The longitudinal modes of the pump laser 110′ may be controlled by dual ring optical resonators 1105 positioned at each end of the linear waveguide that provides the lasing cavity 105. The optical resonators 1105 (shown as SiₓN_{y} dual ring resonators) are configured to reflect the light of the first (fundamental) frequency of the laser 110′, and thus, function as wavelength-selective “mirrors” (with dimensions that determines the specific longitudinal mode frequencies) for single frequency laser feedback without the use of facets, coatings, diffraction gratings, and/or other reflective elements. The optical resonators 1105 or reflectors are not nonlinear optical elements 120, but rather are elements of the active pump laser 110′ (e.g., configured for reflectivity for 400 nm to 480 nm light). That is, the laser diode 110′ includes the double-ring elements as wavelength selective mirrors to form the lasing cavity 105.

As shown in the graphs of FIG. 11B, because the SHG/SFG optical cavity 125 also couples to the laser cavity, the vernier frequency effect provides for selection of only a subset of possible longitudinal modes supported by the laser 110′. It is noted that the SHG/SFG (e.g., 220 nm) light can couple back into the lasing cavity 105 from the nonlinear optical element 120 cavity (i.e., coupling may be in the forward/output direction and in reverse), and that the optical resonators 1105 (the SiₓN_{y} mirrors) may not be 100% reflective, such that there may be losses at the ends of the lasing cavity 105.

Still referring to FIG. 11, the output coupling element 130 is implemented as a waveguide configured to provide a lower cutoff frequency such that the fundamental wavelength (e.g., 440 nm) light is confined within the waveguide but the SHG/SFG wavelength (e.g., 220 nm) light can escape. By selectively outcoupling light from the nonlinear optical element 120, the Q of the ring-shaped optical cavity 125 at SHG/SFG wavelengths may be kept low. In some embodiments, the output coupling element 130 may be configured to semi-continuously outcouple the SHG/SFG wavelength light, for example, where the efficiency of the output coupling element 130 is particularly high. In some embodiments, the output coupling element 130 may conformally extend along at least a part of the nonlinear optical element 120 (e.g., may wrap partially around the ring), such that the SHG/SFG wavelength light may be semi-continuously extracted, which can thereby allow for relaxed (or no) phase matching requirements.

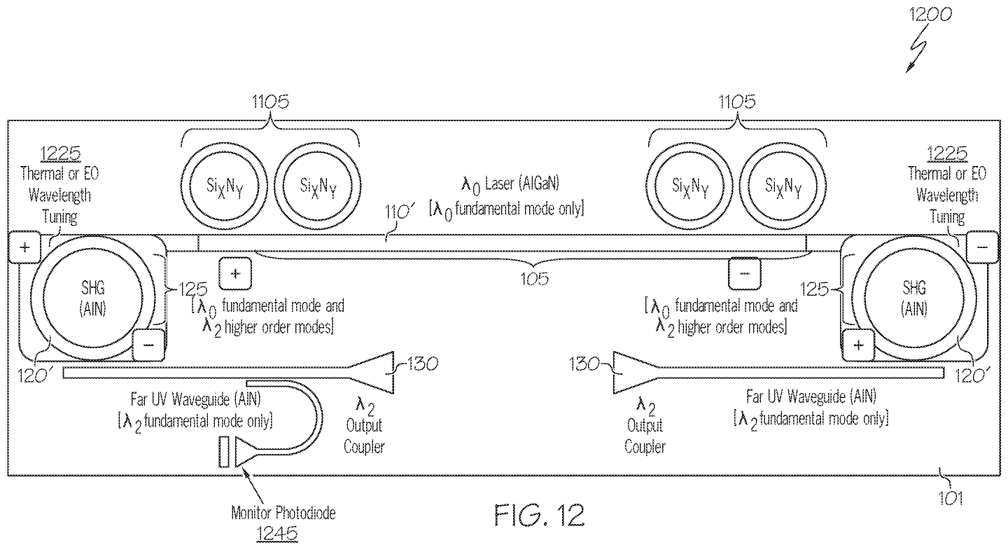

FIG. 12 is a schematic top view illustrating an example combination of various elements of a UV light source 1200 including nonlinear optical elements 120 coupled to an extra-cavity portions of a light emitting element 110, in combination with ring resonators 1105, tuning mechanisms 1225, output coupling elements 130 configured for selective light extraction, and an output monitor element 1245 according to some embodiments of the present disclosure. In FIG. 12, the nonlinear optical element 120 is implemented by multiple (e.g., first and second) SHG/SFG elements 120′ (e.g., two AlN ring resonators) positioned adjacent respective first and second ends of a lasing cavity 105 (shown as including an AlGaN waveguide).

The first and second SHG/SFG elements 120′ are arranged relative to the lasing cavity 105 in an external-cavity-tap configuration, where the light of the fundamental wavelength (e.g., 440 nm) is output from the laser 110′ at the respective first and second ends of the lasing cavity 105, propagating in a single direction at each end. As the laser 110′ is configured for light emission at both ends, the first and second SHG/SFG elements 120′ may be identical (or similar). Providing the first and second SHG/SFG elements 120′ at respective ends 105a, 105b of the lasing cavity 105 may reduce coupling of the SHG/SFG light 121′ back into the lasing cavity 105, such that a greater (or maximum) fraction of light (e.g. approaching 100%) can be coupled into the SHG/SFG elements 120′ (in comparison to the intra-cavity-tap configuration).

The nonlinear optical elements 120 may be arranged adjacent to the opposing ends of the lasing cavity 105 such that coupling between the laser 110′ and the nonlinear optical elements 120 may be in-plane (i.e., along one or more directions parallel to a surface of a substrate 101), and/or may be at least partially stacked on the laser 110′ (e.g., at least partially overlapping the ends of the lasing cavity 105 in the vertical direction, normal to the surface of the substrate 101) such that coupling between the laser 110′ and the nonlinear optical elements 120 may be in a vertical direction (i.e., perpendicular to the surface of the substrate 101). In such a vertically overlapping arrangement, most or all of the fundamental wavelength light 111 that is not reflected back by the double ring optical resonator elements is coupled into the nonlinear optical elements 120 (e.g., the AlN ring resonators), which can improve overall device efficiency. Also, little to none of the SHG/SFG light 121′ may be coupled back into the lasing cavity 105, which can also improve efficiency.

One or more mirror elements (shown as SiₓN_{y} optical resonators 1105) are provided at or near the ends of the lasing cavity 105. The SHG/SFG light 121′ may be coupled (in-plane or vertically) from the first and second SHG/SFG elements 120′ into respective output waveguides, and output by respective output coupling elements 130 at respective ends of the output waveguides. The output coupling elements 130 may be facets, gratings, or other optical elements configured to outcouple the SFG/SHG light 121′ in a direction that is substantially normal to the surface of the substrate 101 (or otherwise out of the plane shown in the illustrated plan view) to provide surface emission.

FIG. 12 also illustrates at least one tuning mechanism 1225 that is configured to adjust one or more operating characteristics of the nonlinear element based on the light output from the light emitting element 110. The tuning mechanism(s) 1225 may include thermal heaters, electro-optic (EO) devices, and/or other devices used to alter a thermal, electrical, and/or optical characteristics of the nonlinear optical elements 120 (e.g., the AlN ring resonators) to more closely match or correspond to the emission wavelength of the light output from the light emitting element 110, which may vary or drift depending on the operating environment (e.g., with changes in temperature) and/or manufacturing tolerances. For example, a wavelength tuning mechanism 1225′ may be provided by a thermal or electro-optic element that is configured to adjust the resonance of the nonlinear optical element 120 to correspond to the output of the laser 110′. In some embodiments, the wavelength tuning mechanism 1225′ may be a gold or other thermally conductive metal plate or element that is configured to alter the resonant wavelength responsive to heating. In another example, the wavelength tuning mechanism 1225′ may include one or more thermoelectric cooling (TEC) elements that are configured to alter the resonant wavelength responsive to cooling.

Such wavelength tuning mechanisms 1225 may be implemented to allow for imperfections or variations in the output of the laser 110′, for example, wavelength drift with changes in operating temperature, and may be similarly used in any of the embodiments described herein. Also, while shown by way of example as being used to adjust characteristics of the nonlinear optical elements 120, the tuning mechanisms 1225 may be similarly used to adjust characteristics of other elements of UV light sources (such as the one or more (SiₓN_{y}) wavelength-selective optical resonators 1105 and/or the light emitting element 110 itself) to more closely match or control the operating characteristics of the light emitting element 110 and the nonlinear optical elements 120. For example, the wavelength-selective optical resonators 1105 may include respective tuning mechanisms 1225 to be adjusted such that the reflected laser emission 111 matches the characteristics of the nonlinear optical elements 120 for second harmonic or sum frequency generation. Wavelength tuning (thermal or electro-optic) as described herein may also be used to compensate for variations in the coupling gaps (e.g., between the laser 110′ and the nonlinear optical elements 120, and/or between the nonlinear optical elements 120 and the output waveguides), and/or the refractive index of one or more optical elements. That is, the tuning mechanisms 1225 may be used with multiple optical elements described herein.

FIG. 12 further illustrates a monitor element 1245 that is configured to measure a property (e.g., detect a power level) of the output light 131 and generate a feedback signal to a controller that is configured to operate the light emitting element 110 and/or the tuning mechanism 1225. For example, the monitor element 1245 may be implemented as an integrated photodiode that is configured to detect or monitor the field strength or power of the SHG/SFG light 121′ output in order to provide feedback signals to other system components such as the laser drive and/or the tuning elements. One or more monitor photodiodes 1245 may similarly be integrated at various positions in the UV light source 1200 to monitor the light output from the light emitting element 110 and provide feedback signals to one or more controllers. Such monitor elements 1245 may be similarly used in any of the embodiments described herein.

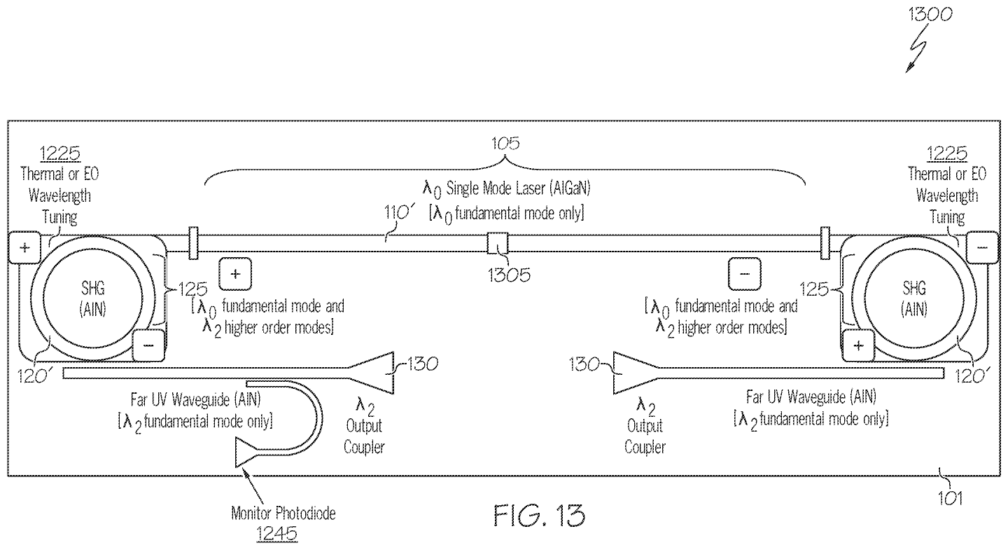

FIG. 13 is a schematic top view illustrating an example combination of various elements of a UV light source 1300 including nonlinear optical elements 120 coupled to an extra-cavity portions of a light emitting element 110 including a saturable absorber element 1305, in combination with tuning mechanisms 1225, output coupling elements 130 configured for selective light extraction, and an output monitor element 1245 according to some embodiments of the present disclosure. It will be understood that these and other illustrated implementation are by way of example, and that embodiments of the present disclosure may include various other combinations of the illustrated (and other) components.

The UV light source 1300 of FIG. 13 may be similar in some aspects to the UV light source shown in FIG. 12, and thus, description of similar elements will not be repeated for brevity. However, in FIG. 13, the light emitting element 110 (e.g., the AlGaN pump laser 110′) may include end-mirror facets or other elements to define the lasing cavity 105, without use of the dual ring reflectors. In addition, a saturable absorber element 1305 is integrated or otherwise provided in the lasing cavity 105. The saturable absorber 1305 is configured to induce the laser 110′ to operate in a (passive or active) mode-locked manner or otherwise generate the light of the fundamental wavelength as a plurality of light pulses. In particular the saturable absorber 1305 is configured to generate higher power pulses, which may increase SHG/SFG conversion efficiency (e.g., by exploiting quadratic nonlinearity in the SHG/SFG elements 120′). The saturable absorber 1305 may be substantially transparent at higher lasing intensities, which may cause the lasing cavity 105 to preferentially support modes in which the optical power is pulsed. It is understood that the embodiments here can include any type of saturable absorber material configured based on the desired output wavelengths of the light emitting element 110 (e.g., at the 400-480 nm wavelengths), or may include artificial saturable absorbers such as Kerr Lens structures with an aperture.

As in other embodiments, the SHG/SFG elements 120′ are implemented as resonant cavities (shown in FIG. 13 as AlN-based ring-shaped cavities) that are overcoupled (e.g., near 100% collection) to the output of the pump laser 110′ at opposing ends of the lasing cavity 105 (i.e., in an external cavity-tap configuration). Respective wavelength tuning mechanisms 1225′ are used to adjust the operating characteristics of the SHG/SFG elements 120′ (e.g., the AlN ring resonators) to match the emission wavelength of the laser 110′. Also, one or more output monitors 1245 (e.g., photodiodes) provide feedback signals (e.g. to drive controllers/electronics) to adjust the drive current and/or the tuning elements based on the detected light output from the light emitting element 110 (i.e., the fundamental light) and/or the output coupling elements 130 (i.e., the SHG/SFG light 121′). More generally, elements, mechanisms, arrangements, and/or other configurations described herein with respect to one embodiment may be combined with those of other embodiments unless otherwise noted.

Further embodiments of the present disclosure are described below with reference to one or more design features that are configured to improve yield of emitters that provide far-UVC light output 131′ using cavity-enhanced SHG. These embodiments are described herein by way of example with reference to a light emitting element 110 implemented as a laser diode 110′ that is configured to emit light having a predetermined wavelength, e.g., 440 nm light. The configuration of the laser 110′ may be in accordance with any of the embodiments described herein, including but not limited to ridge or buried-slab waveguides; mirror elements on ends thereof; distributed feedback (DFB) configurations; and/or double ring reflective elements at respective ends 105a, 105b thereof.

FIG. 14 is a schematic top view illustrating an example combination of various elements of a UV light source 1400 including a plurality of different or non-identical nonlinear optical elements 120 in ring cavity configurations with respective output coupling elements 130 according to some embodiments of the present disclosure. As shown in FIG. 14, light emission from the laser diodes 110′ is coupled directly into a linear (in these examples, unidirectional) input waveguide as the input coupling element 115 through which the fundamental wavelength light 111 propagates in a single (or small number of) mode(s). The input coupling element 115 (at the laser output) may be a 440 nm input waveguide or single mode fiber. Nonlinear optical elements 120 are provided by ring shaped SHG/SFG elements 120′ (shown as a plurality of AlN ring resonators; but more generally, a plurality of nonlinear optical elements 120) along an edge or side of the length of the input waveguide 115. The ring shaped SHG/SFG elements 120′ may be fabricated from various nonlinear optical materials (in these examples, AlN, although other embodiments may use different materials). The ring shaped SHG/SFG elements 120′ are configured to be doubly resonant at both the fundamental and the higher order frequencies or wavelengths (e.g., 440 nm and 220 nm), and the ring design may be configured to provide high Q for a single mode at both the fundamental frequency ω₁ or wavelength (e.g., 440.0 nm) and the higher order frequencies ω₂ or wavelengths (e.g., 220.0 nm).

In particular embodiments, it may be critical that the waveguide is configured to support modes at wavelengths of the two modes supported that are related by a factor of 2.0 exactly, in order to yield improved or best possible SHG conversion efficiency. The doubly resonant cavities of the ring shaped SHG/SFG elements 120′ may be configured to have a gap between edges thereof and the edge of the adjacent input waveguide, where the size of the gap may be configured and carefully controlled to improve or maximize coupling of the 440 nm light from the input waveguide into the ring shaped SHG elements. In addition, on the other side (as shown, on the bottom) of the ring shaped SHG elements, one or more linear output waveguides are fabricated and configured to collect 220 nm light outcoupled therefrom. In some embodiments, the respective output waveguides (in the illustrated embodiment, one per ring shaped SHG/SFG element 120′) are configured such that only the desired wavelength of light (the SHG/SFG frequencies or wavelengths, e.g., 220 nm) is supported (and not the fundamental frequency or wavelength, e.g., 440 nm) because the output waveguide's critical frequency is greater than the fundamental frequency.

Without wishing to be bound by theory, some advantages of providing a plurality of nonlinear optical elements 120 may be explained as follows. Although the design of the ring shaped nonlinear optical elements 120 may be such that a single or respective cavity can deliver very high SHG conversion efficiency, the manufacturing tolerances for the radius, the coupling gap(s) and the losses of each ring shaped nonlinear optical elements 120 may be difficult to maintain from one emitter to another. Accordingly, multiple, nominally identical (or substantially similar) nonlinear optical elements 120 along the edge of the waveguide may be provided for redundancy, where each of the nonlinear optical elements 120 may have slightly different dimensions (e.g., radius R, supported frequency ω, gap to waveguide, etc.). As such, even if only a single one of the plurality of nonlinear optical elements 120 provides sufficient SHG efficiency, the overall device (laser+waveguide(s)+nonlinear optical element 120) may operate as intended.

That is, as fabrication tolerances of structures or elements of the UV light source 1400 may present challenges, some embodiments may include a plurality of variants of SHG/SFG elements 120′ to address differences in manufacturing tolerances. In particular, as the pump light 111 is coupled out of the laser 110′ and propagates down the input waveguide, the geometry (including dimensions and shapes) of one or more (or each) of the SHG/SFG elements 120′ may be different, either by design or due to manufacturing variations. With different geometries, each SHG/SFG element 120′ may have a different amount or level of coupling to the waveguide, or may have different resonant frequencies, such that one or more of the plurality of SHG/SFG elements 120′ may be particularly well matched (in terms of wavelengths of operation) to the pump laser 110′, while one or more others of the plurality of SHG/SFG elements 120′ may not be as well matched to the pump laser 110′. As such, only a subset of the plurality of SHG/SFG elements 120′ may contribute the majority of the SHG/SFG light 121′ generated by the UV light source 1400. Alternatively, with changes in temperature or operating conditions, one or more SHG/SFG elements 120′ may come into and out of ideal matching with the pump wavelength (i.e., due to variations in operating characteristics over the time or duration of operation of the UV light source. Overall, redundancy provided by the plurality of variants of the SHG/SFG elements 120′ may contribute to the robustness of the overall performance of the UV light source 1400.

In other words, to address challenges with respect to manufacturing tolerances, a plurality of nonlinear optical elements 120 may be intentionally fabricated (e.g., along a length of an input coupling element 115 or lasing cavity 105) with one or more different dimensions, shapes, or even materials, such that at least one of the nonlinear optical elements 120 might have the desired dimensions to yield high conversion efficiency with respect to the fundamental wavelength of the light output from the light emitting element 110. The remaining (i.e., non-conforming) nonlinear optical elements 120 may be unused if they have either poor coupling to the input waveguide or they do not provide sufficiently high SHG/SFG efficiency, and any fundamental wavelength light 111 that is coupled into the non-conforming ring shaped nonlinear optical elements 120 may be returned or outcoupled back into the input waveguide. Or the non-conforming nonlinear optical elements 120 may be intentionally removed, destroyed or disabled by some additional process step after fabrication.

Because the overall area or footprint of the UV light source 1400 may be dictated by the length of the laser 110′ and the width (i.e., along a direction perpendicular to the length of the laser 110′ or waveguide) of a respective nonlinear optical element 120, the inclusion of “extra” or unused nonlinear optical elements 120 of nominally the same or similar configuration may not require substantially more area on the chip. That is, the use of multiple nonlinear optical elements 120 may have relatively little cost, particularly when the SHG/SFG element 120′ sizes are small (e.g., a fraction of the length) in comparison to the dimensions of the pump laser 110′ and/or waveguide. However, including the additional nonlinear optical elements 120 can increase the likelihood of desired device functionality and/or achieving high conversion efficiency. In this way, providing a plurality of ring shaped or other nonlinear optical elements 120 with one or more different dimensions, shapes, and/or materials along a length of the waveguide can lead to higher device yields than may conventionally be possible, with little to no size penalty.

More generally, in the example of FIG. 14, respective ones of the nonlinear optical elements 120 may have different dimensions, shapes, and/or materials, and the output coupling element 130 may include a plurality of output coupling elements 130 that are respectively configured to selectively outcouple far-UVC light 121′ as output light 131′ from the respective ones of the nonlinear optical elements 120. As noted, variations of the embodiments described herein may be implemented with a similar layout or configuration, but including (a) different types of laser 110′ that is used to pump the system or the material used for the laser 110′, such as GaN, AlGaN, InGaN, etc.; (b) different materials used for the nonlinear optical elements 120, such as AlN, Li Niobate, BBO, and/or other nonlinear materials; (c) different shapes or designs of the waveguides themselves (e.g., some embodiments may use ridge waveguides with flat edges while others may use slabs of high index material with the guiding provided by a lateral variation in the cladding, etc.); (d) different substrates such as AlN, GaN, Si, Sapphire or other materials; (e) different methods of fabrication in order to heterogeneously integrate the various components (e.g., monolithic fabrication using epi regrowth, wafer bonding, and/or microtransfer printing). Any or all of the various combinations of embodiments surrounding the concepts described herein are included in the above embodiment using multiple nonlinear optical elements 120 along the same waveguide.

Further embodiments of the present disclosure may likewise provide multiple nonlinear optical elements 120 along one side or opposing sides of an input waveguide (at the laser output), but may remove or omit the respective output waveguides (shown in FIG. 14 as providing the SHG/SFG light 121′ from the nonlinear optical element 120 to the output coupling elements 130). Rather, the SHG/SFG light 121′ generated by the nonlinear optical elements 120 may escape by radiative bending losses from the ring-shaped nonlinear optical elements 120. In general, the smaller the bending radius, the higher the radiative losses from a ring shaped waveguide. The ring-shaped nonlinear optical elements 120 may thus be configured to account for the radiative bending losses when considering the radius thereof. However, in contrast to the embodiment shown in FIG. 14 (which maintains low radiative losses relative to the coupling of SHG/SFG light 121′ out through the bottom output waveguides as output light 131′), further embodiments may be configured to emit some or up to all of the SHG/SFG light 121′ as output light 131′ via the bending radius of the respective ring-shaped nonlinear optical elements 120. Without wishing to be bound by theory, some advantages of removing the respective output waveguides and relying on radiative bending emission to collect the SHG/SFG light 121′ may include reduced manufacturing complexity, by removing at least one component of the overall device (e.g., the 220 nm output waveguide(s)) whose dimensions may otherwise require rigorous precision with respect to dimensions and positioning relative to the nonlinear optical element 120 (s).

That is, while FIG. 14 illustrates the use of multiple SHG cavities in an “external-cavity-tapped” configuration, and with output waveguides provide to couple the SHG light out, other embodiments may vary by providing “intra-cavity-tapping” of the pump laser 110′, and/or radiative or scattering output of the SHG/SFG light 121′ (instead of the use of a waveguide). Furthermore, these embodiments may use nonlinear optical elements 120 that are not ring cavities, and instead are some other kind of whispering gallery mode cavity or even some kind of linear cavity.

The omission of the output waveguide element(s) for collection of the SHG/SFG light 121′ outcoupled from the nonlinear optical elements 120 indicates that the output light 131 may be emitted laterally (i.e., in-plane light emission with respect to the above plan view), rather than surface emission in a direction perpendicular to the plane in the plan view shown above (or otherwise with some out of plane component). As such, in some embodiments, one or more angled reflectors can be integrated around the exterior (e.g., the circumference) and/or interior of the ring. Such angled reflectors (not shown) may extend around and/or within a circumference or perimeter of the nonlinear optical elements 120 in plan view. For example, in cross sectional view, the angled reflector may be triangular-shaped reflectors on opposing sides of the nonlinear optical elements 120. It will be understood that some out-of-plane emission components might also be incorporated into the SHG/SFG light 121′ Poynting vector (propagation direction) by the shape of the side walls of the ring shaped nonlinear optical elements 120 (which may be trapezoidal in cross section). That is, the sidewalls of the nonlinear optical elements 120 may have a substantial angle in cross section, such that the light may be emitted with some upward/out of plane component. In other words, the nonlinear optical element 120 waveguide sidewall angle may be controlled or otherwise configured to optimize desired light emission in some embodiments.

It will be understood that the concept of SHG/SFG emission via bending losses as described in the above embodiment may be similarly implemented in any of the embodiments described herein. For example, any of the embodiments including ring shaped nonlinear optical elements 120 as described herein may be configured such that the light generated by the nonlinear optical elements 120 may escape by radiative bending losses from the ring-shaped nonlinear optical elements 120 (e.g., by omitting the output coupler(s) and using one or more angled reflectors for light extraction in the desired direction).

Further embodiments of the present disclosure may likewise provide multiple nonlinear optical elements 120, but in an intra-cavity configuration to receive input light directly from the lasing cavity 105 at one side thereof, in combination with one or more output waveguides (e.g., one per nonlinear optical element 120) along the other side thereof. In particular, further embodiments may similarly include the SHG/SFG light 121′ output waveguides for outcoupling the SHG/SFG light 121′ (e.g., at 220 nm) from each ring resonator as shown in FIG. 14, but instead of introducing the fundamental wavelength light 111 to the nonlinear optical elements 120 by means of a unidirectional wave from a laser 110′, the nonlinear optical elements 120 may be arranged alongside a waveguide that provides the lasing cavity 105 of the laser 110′. That is, while shown in FIG. 14 as including an input coupling element 115 implemented as an input waveguide, the input coupling element 115 may be omitted and light from the light emitting element 110 can be coupled directly into the nonlinear optical elements 120 from within the cavity of the (active) laser 110′ itself, rather than from a separate passive input waveguide. Such embodiments may be more complex with respect to design of the laser 110′ itself (the presence of one or more rings represents a loss term in the energy balance of the laser 110′) but may be advantageous in that higher intensity pump power (e.g., at 440 nm) may be provided. Remaining aspects of this embodiment may be the same as or similar to previous embodiments, including variations with respect to materials, substrates, and/or and fabrication methods as noted herein.

Further embodiments of the present disclosure may likewise provide multiple nonlinear optical elements 120 configured to receive input light at one side thereof in combination with one or more output waveguides (e.g., one per nonlinear optical element 120) along the other side thereof. However, the nonlinear optical element 120 may be implemented with shapes other than rings (for example, other rotationally symmetric shapes, such as disk (e.g., microdisks) or sphere (e.g., microspheres) shapes). The modes supported by such structures may be different than that of a ring-shaped nonlinear optical element 120, but the overall concept and benefits of use of a plurality of resonators remain the same as previous embodiments.

Further embodiments of the present disclosure may similarly include multiple nonlinear optical elements 120 configured to receive input light at one side thereof in combination with one or more output waveguides (e.g., one per nonlinear optical element 120) along the other side thereof, with the nonlinear optical elements 120 (e.g., ring resonators) coupled directly to the intra-cavity region 105i of the lasing cavity 105 of the laser diode 110′. However, the laser diode 110′ (to which the plurality of ring-shaped nonlinear optical elements 120 is coupled) may be implemented as a ring laser, rather than a linear ridge laser. Coupling to a ring laser may be advantageous in terms of the type of mode to which coupling can be realized. Coupling to a ring laser as the light emitting element 110 may also be configured for coupling only to modes that propagate in a single direction (as opposed to multiple directions, as may be obtained from a linear laser which contains a standing wave).

Further embodiments of the present disclosure may include at least one nonlinear optical element 120 configured to receive input light at one side thereof, in combination with at least one output waveguides (e.g., one per nonlinear optical element 120) along the other side thereof. However, optical coupling between elements may be realized at least in part by lateral overlap of the nonlinear optical element 120(s) with the components for input and/or output light coupling, such that at least two of the components (e.g., the light emitting element 110, the input coupling element 115, the nonlinear optical element 120, or the output coupling element 130) are not in the same plane, also referred to herein as multi-layer integration.

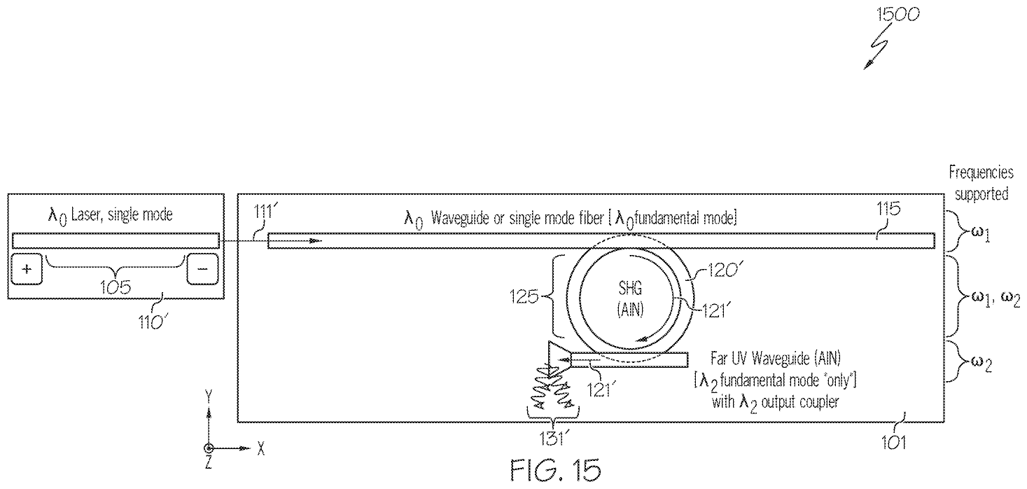

FIG. 15 is a schematic top view illustrating an example combination of various elements of a UV light source 1500 in physically overlapping configurations for multi-layer integration according to some embodiments of the present disclosure. In FIG. 15, the geometry of coupling differs from some embodiments described herein in that respective portions of the SHG/SFG element 120′ physically overlap the input and output waveguides, but are not arranged on a same plane or coplanar surface (i.e., the elements are offset in the vertical or Z direction (in or out of plane) relative to one another).

Advantages conferred by the multi-layer integration arrangement shown in FIG. 15 include that the critical dimensions through which optical coupling occurs are in the vertical or Z-direction (i.e., the out of plane direction in the illustrated plan view), which can be easier to control by many fabrication methods. It will be understood that the vertical overlap of two or more components as shown in the embodiment of FIG. 15 may include any and all methods of integrating or manufacturing multiple components such that two or more do not share the same plane. Example methods may include wafer bonding, microtransfer printing, microassembly, self-assembly, and/or polishing and epitaxial regrowth. Additional variations of vertically overlapping elements beyond that shown in FIG. 15 may also be used for coupling. Such coupling configurations may include, but are not limited to, vertical overlap evanescent wave coupling, lateral evanescent coupling, use of gratings to direct light in or out of plane of the waveguide, butt (end-to-end) coupling, and/or use of tapered element shapes (e.g., waveguides with non-uniform thicknesses) to enhance coupling. More generally, UV light sources as described herein may include two or more of the light emitting element 110, the input coupling element 115, the nonlinear optical element 120, and the output coupling element 130 overlapping in a direction that is perpendicular to a surface of a substrate 101 having the light emitting element 110, the nonlinear optical element 120, and the output coupling element 130 thereon.

Further embodiments of the present disclosure may include various coupled ring configurations, which may extend the vernier frequency selection strategy through the use of an intermediate cavity (e.g., a ring- or other-shaped cavity between the lasing cavity 105 and the optical cavity 125 of the nonlinear optical element 120) to select only a single mode over an even larger free spectral range (FSR). Doing so may help concentrate energy from the pump laser into a single mode that is doubled and thus increase efficiency.

Some embodiments may be configured to provide the ability to switch coupling on-and-off by providing an electro-optic or thermo-optical material between two rings (to shift the index electrically or thermally). For example, the electro-optic or thermo-optical material may be provided between a ring laser and a ring-shaped nonlinear optical element.

Some embodiments may include a saturable absorber in the ring laser to induce pulsed modes.

Some embodiments may include one or more secondary rings as ‘filters’ to provide wider free spectral range and matching specific ring mode to specific SHG/SFG ring mode. For example, at least one passive oscillator may be provided as a secondary ring that receives light outcoupled from the ring laser and outcouples a subset of the light to a nonlinear optical element for secondary harmonic generation.

Some embodiments may include multiple nonlinear optical elements that are arranged partially or substantially around a periphery of one laser. For example, multiple ring-shaped nonlinear optical elements 120 are provided around a circumference of a single ring laser.

Some embodiments may include one or more secondary rings as filters that are arranged partially or substantially around a periphery of one laser, within a larger nonlinear optical element. For example, a large radius ring-shaped nonlinear optical element extends around a ring laser, which may be filtered in some embodiments by one or more ring-filter oscillators to allow mode selection from SHG ring which has high mode density.

Some embodiments may include multiple ring lasers per nonlinear optical element. For example, multiple ring lasers may be arranged around a periphery (or circumference) of a ring-shaped nonlinear optical element. Due to coupling between rings, the ring lasers may all be forced to same phase or mode. In some embodiments, the ring lasers may be turned on or activated sequentially, allowing the first ring laser to set the phase for the remaining ring lasers. Some embodiments may include a wavelength tuning mechanism configured to provide localized temperature or electric field tuning of each ring laser independently, which can provide another degree of freedom.

As noted above, while some conventional designs of UV light sources may be handicapped by coupling losses between the active and passive components and/or conversion efficiencies, embodiments of the present disclosure may provide higher nonlinear conversion efficiencies by use of optical cavities 125 to increase the number of passes that the pump laser 110′ makes through the material (effectively recycling unconverted pump light 111). These benefits have been demonstrated at other wavelengths. For example, optical microresonators fabricated from AlN have demonstrated over 17,000%/W SHG/SFG conversion efficiency (up to 10% absolute conversion efficiency for 10 mW input) and 180%/W2 third harmonic conversion efficiency, albeit using a 1540 nm fundamental wavelength [9-12]. These results demonstrate that AlN has a sufficiently high nonlinear response (4 pm/V vs. 7 pm/V) to deliver very high conversion efficiency, in particular when cavity-enhancement is also used to increase or maximize the intensity of the fundamental wavelength.

Further embodiments of the present disclosure may provide ways of controlling the far field pattern of the output light 131 that is outcoupled from nonlinear optical elements 120 described herein (e.g., the far-UVC light 121′). For some applications, control of the spatial distribution of irradiance over an area (or equivalently, the angular distribution of radiant intensity over some field) of illumination may be critical to performance. Indeed, visible illumination products support an entire industry dedicated to shaping and sculpting the pattern of illumination. For UV applications, there may be similar need for control of this “far field pattern”, for example, to provide germicidal efficacy for the far-UVC output light 131′, which may depend on spreading the germicidal UV across a region of application in an optimal manner.

Some embodiments the present disclosure may include an output coupling element 130 implemented as a second order diffraction grating that is configured to couple the light out of an in-plane PIC and project it over a range of angles surrounding the normal surface vector. The design or configuration of the diffraction grating may provide some ability to modify how wide of an angle the light is spread over as well as the uniformity of the radiant intensity within the range of emission angles.

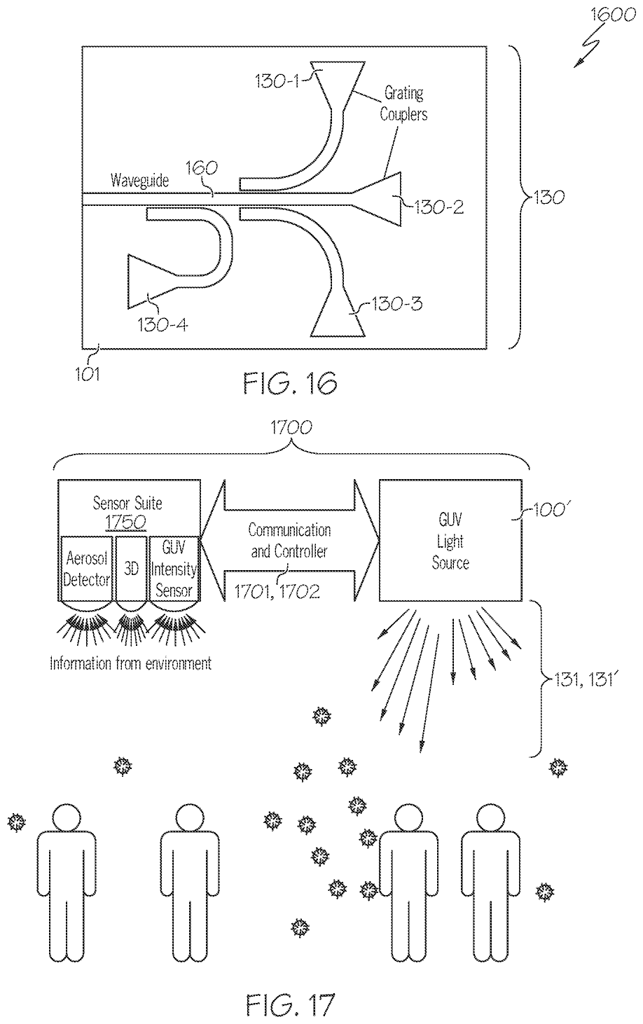

In further embodiments, the photonic integrated circuit may include structures that are configured to divide the light generated on the chip into multiple channels, each of which has its own output coupling element 130 which may or may not include a second order diffraction grating. FIG. 16 is a schematic top view illustrating output coupling element configurations 1600 of a UV light source configured to provide a desired far field emission pattern according to some embodiments of the present disclosure.

In FIG. 16, the output coupling element 130 is implemented as a plurality of output coupling elements 130-1, 130-2, 130-3, 130-4 that are configured to outcouple the far-UVC light 121′ as output light 131 in respective directions (e.g., at various angles in-plane or out of the page or substrate 101 as depicted), to provide the output light 131 with a desired far field pattern. One or more waveguides 160 may be coupled between the nonlinear optical element 120 and the respective output coupling element 130, and the output light 131 may be a split beam by respective waveguides and output coupling elements 130 to provide respective channels for light output. The multiple output coupling elements 130 may be configured to direct the light upward (e.g., away from a surface or substrate 101), but in directions that are not necessarily or perfectly normal (e.g., not necessarily at 90 degrees) to the surface or substrate 101. By centering or providing the emission pattern of the termination of each channel to provide the output light 131 (e.g., the far-UVC light 121′) into different directions (coherently or incoherently), a combined overall light pattern can be designed to the requirements of a desired application. The number of channels for which this configuration may be used could be as few as one or two, but the upper bound is limited only by practicality of PIC design (e.g., thousands or more).

Two specific subclasses of multiple output channel configurations as described herein include arrays of UV light sources, and coherent light combination. In an array of UV light sources, the light output of any one UV light source may or may not be divided into multiple output couplers. However, because the devices are meant to be operated as part of a larger array of nominally identical devices, the output coupling element 130 (s) of each individual UV light source within the array may be individually modified such that the combined far field pattern of the overall array meets a desired specification.

In coherent combination of light, the individual UV light sources may have their respective light output divided into at least two different channels. Because the output light 131 (e.g., the far-UVC light 121′) emitted from each of two or more channels originate from the same coherent light source (e.g. the visible light 111′ output from the laser 110′), the output light 131 from the respective channels may maintain a fixed phase relationship. As such, the far field emission pattern generated by the respective emission channels (one per UV light source) may be subject to coherent effects (similar to that used for optical beam steering). In other words, some embodiments may take advantage of coherent combinations of output from respective channels of multiple individual UV light sources in order to obtain a desired emission pattern. When multiple UV light sources are operated as a very large array or as an array with distinct or different output coupling element configurations, however, it may be unlikely that phase coherence can be maintained between the UV light sources, so far field patterns generated by the collective light output across an array may include an incoherent combination of optical fields.

Embodiments of the present disclosure may differ from some conventional designs in several ways. For example, some embodiments of the present disclosure integrate active and passive components on the same chip (e.g., using components of the same material systems, such as nitride based materials) such that the optical losses between devices are reduced or minimized. In addition, some embodiments of the present disclosure may specifically target conversion from 440 nm to 220 nm with a focus on conversion efficiency, in contrast to designs that may attempt to fabricate coherent, polarized laser (beams) with narrow linewidth, which may not be necessary for some applications. Also, in contrast with previous demonstrations of SHG to generate 220 nm light, PICs in accordance with some embodiments of the present disclosure leverage resonant cavity enhancement to increase or maximize the intensity of the fundamental wave and thus increase or maximize efficiency. The output light provided by embodiments of the present disclosure may be collimated or non-collimated, coherent or incoherent, and emitted as a beam or as distributed emission. Some embodiments may use (but are not limited to) one or more of the following technology elements, in various combinations: wavelength conversion using nonlinear optics (SHG/SFG); use of AlN-based nonlinear optical elements 120 for wavelength conversion; selective outcoupling of far-UVC wavelengths; use of waveguides, including AlN-based waveguides or PICs; use of optically resonant microcavities; monolithic integration of active and passive components; and light output that is free of the fundamental wavelength of the light emitting element 110.

Embodiments of the present disclosure as described herein may thereby reduce cost and increase the (power) efficiency for producing far-UVC light 121′, which may be advantageous in providing a cost-competitive source of disinfecting light that can be widely deployed to combat airborne (and surface) pathogens. Moreover, by providing UV light emission in the far-UVC range (e.g., from about 200 nm to about 240 nm), embodiments of the present disclosure can be used to actively eliminate pathogens from the air while people are present, in contrast to conventional use of UV wavelengths for disinfectant purposes in wavelength ranges that are harmful to humans (e.g., from greater than about 240 nm to about 400 nm).

Further embodiments of the present disclosure provide devices configured to generate electromagnetic radiation in the far-UVC spectrum to provide germicidal effects, while also complying with human safety regulations and requirements. Germicidal light sources configured to operate in the far-UVC wavelength range may be advantageous in that (i) the rate of disinfection of pathogens may be higher, and (ii) from a human safety perspective, acceptable levels of irradiation may be higher (and perhaps infinite or limitless) as compared to the remainder of the wavelengths in the UV spectrum.

In light of safety regulations and/or concerns, it may be advantageous to operate GUV light sources only when necessary and/or at power levels, duty cycles, and/or spatial illumination patterns that are optimized for minimizing risk of airborne pathogen transmission. Achieving such operation may require detection of operating conditions and/or other information in real time.

Embodiments of the present disclosure described herein can provide real-time, actionable information to a GUV light source by integrating sensors into the GUV system operation, either physically or by way of communication networks. In particular, some embodiments of the present disclosure provide a sensor feedback-based “smart” illumination device that includes a GUV light source communicatively coupled to sensors of various types, which are configured to feedback information to a controller of the GUV light source to allow for algorithmic decision making and optimized operation.

Integration of sensor(s) and GUV light sources into a single device may be advantageous in terms of the capability and scope of operation of GUV illumination products, allowing detected operating conditions to be provided to a controller in real time, allowing for control of the operation of the GUV light source in accordance with the detected operating conditions. GUV irradiation and illumination may thereby be optimized, i.e., with respect to increasing or maximizing the effectiveness of the GUV in terms of ability to disinfect while reducing or minimizing any overall GUV optical output in the interest of remaining within safety limits, prolonging GUV lifetime, and reducing or minimizing impact of UV light on the surrounding environment.

In contrast, some conventional GUV systems may not be configured to detect or control operations based on existing operating conditions. Rather, such conventional GUV systems may be operated with a limited, small number of states, typically “on” or “off” irradiation states. Moreover, such conventional GUV systems may require manual intervention in order to modify the operating condition of the GUV light source. Some GUV systems are driven by autonomous robots that are used to disinfect surfaces inside an enclosed room. While these autonomous robots may employ sensors in conjunction with the operation of the UV light, the sensors are typically directed to controlling the operation of the autonomous robot, rather than optimization of the GUV illumination in a dynamic environment.

Also, while sensors may be conventionally used in combination with typical visible lighting, embodiments of the present disclosure are directed to operation in the UV spectrum where (a) the availability and cost of illumination is scarce and (b) concerns regarding human safety are particularly high. For GUV applications, the types of sensors used and reasons for employing them may be distinct from those of general (visible) lighting applications. For example, sensors that may guide use of GUV lighting may include various forms of air quality sensors (aerosol detectors, pathogen detectors, etc.) in order to judge the degree of need for or effectiveness of GUV illumination including the relative intensity with which the illumination fixtures should be operated. Alternatively or in addition to the use of these sensors, 3D time of flight cameras or other positional sensors that can both detect movement and quantify occupancy levels in a given space may be used to moderate the amount of GUV illumination provided in order to stay within regulatory limits. In either of these cases the distinction from the kind and sophistication of any sensors that are integrated in general lighting is great.

Some elements of embodiments of the present disclosure may integrate a sensor suite with a GUV illumination devices as described herein. FIG. 17 is a schematic block diagram illustrating components of a sensor feedback-based “smart” illumination device that includes a germicidal UV (GUV) light source communicatively coupled to sensors 1750 that are configured to feedback information to a controller 1701 of the GUV light source according to some embodiments of the present disclosure.

In particular, FIG. 17 illustrates an illumination device 1700 including a GUV light source 100′ configured to generate and emit electromagnetic radiation in the germicidal region of the UV spectrum. In some embodiments, the GUV illumination device 1700 may be configured to provide light emission in the far-UVC spectrum, from about 200 nm to about 240 nm (e.g. at about 222 nm). The GUV illumination device 1700 may include a UV light source (such as the UV light source 100′), a controller 1701, and one or more sensors 1750 configured to detect real-time conditions in an operating environment of the UV light source 100′, and to provide detection signals indicating the real-time conditions to the controller 1701. The controller 1701 is configured to control operation of the light emitting element 110 of the GUV light source 100′ based on the detection signals.

The GUV light source 100′ may be implemented using solid state systems for generating coherent or non-coherent, electromagnetic, non-ionizing radiation in the far-UVC wavelength band, based on nonlinear optical processes and using photonic integrated circuits (PIC), as described above in “Nonlinear Solid State Devices For Optical Radiation In Far-UVC Spectrum” to Fisher, et al., the disclosure of which is incorporated by reference herein. Alternatively, the GUV light source 100′ may be implemented by any of the UV light sources (e.g., 100, 200, 300, etc.) or arrays (e.g., 499, 900) described herein. For example, the GUV light source 100′ may include a light emitting element 110 implemented by a pump laser 110′ (e.g., a Group-III nitride-based laser diode, such as a blue pump laser diode) or light emitting diode (LED) configured to generate visible light 111′, and a nonlinear optical element 120 (e.g., a nonlinear optical crystal) that is configured to receive the visible light 111′ from the light emitting element 110 and generate far-UVC light 121′ of a second frequency based on the visible light 111′ of the first frequency (e.g., based on SHG or SFG). The nonlinear optical element 120 may be optically transparent to wavelengths at or below the desired output wavelength (e.g., the far-UVC wavelength range). An input coupling element 115 (e.g., a continuous waveguide that connects radiation from the pump laser 110′ or LED to the nonlinear optical crystal) may be configured to couple light from the pump laser 110′ into the nonlinear optical element 120. In some embodiments, phase matching may be provided between the SHG/SFG light 121′ and the fundamental (pump) wavelength light 111′. An output coupling element 130 is configured to outcouple the SHG/SFG light 121′ from the nonlinear optical element 120, either selectively or in combination with the visible light 111′ (that is, such the light output includes the far-UVC light 131′ alone, or the far-UVC light 131′ of the second frequency alone, or in combination with the visible light 111′ of the first (fundamental) frequency) as output light 131′. However, it will be understood that embodiments of the present disclosure may be used for sensor feedback-based control of other GUV light sources.

Still referring to FIG. 17, a sensor suite including one or more sensors 1750 of various types are configured to detect real-time conditions in the operating environment of the GUV light source. Communication between the sensor suite and the GUV light source 100′ may be provided by a controller 1701 and/or other communicative coupling. The communication/controller 1701 is configured to provide information obtained by the sensors 1750 back to the GUV device 1700 in order to control the operation of the GUV light source 100′ for light generation 131.

The sensors 1750 are thereby configured to provide information feedback to improve or optimize the operation of the GUV illuminator 1700 for a desired application. The sensors 1750 may also be configured to detect and communicate information for purposes other than operation of the GUV illuminator 1700. Examples of possible sensors 1750 include, but are not limited to, air quality sensors (such as humidity, temperature, VOC, chemical sensors (CO2, CO, etc.), particular matter sensors, and aerosol sensors; biological sensors such as virus or pathogen detectors, etc.; radar sensors, e.g., for assessing distance to objects; 2D camera sensors, e.g., for assessing conditions inside the area of operation including personnel and occupancy; 3D cameras or lidar systems e.g., for measuring distances to objects, occupancy, motion, etc.; irradiation sensors, e.g., for assessing the intensity of GUV irradiation within a field of view over the course of time; and/or passive infrared (IR) or other motion sensors.

The communication channel 1702 between the sensors 1750 and the controller 1701 may be bi-directional, so that information from the GUV light source 100′ can be shared with the sensor suite 1750 in order to obtain more accurate measurements of the environment. That is, the controller 1701 may be configured to control operation of the GUV light source 100′ based on the information or data output from the sensors 1750, and/or to control operation of the sensors 1750 based on the operation and/or light output 131 of the GUV light source 100′.

Furthermore, the components (e.g., 110′, 1701, 1750) of the GUV illumination device 1700 may or may not be integrated within a same housing. For example, it will be understood that one or more sensors 1750 of the sensor suite and GUV light source 110′ need not be contained within the same physical housing, and/or need not even be collocated. More generally, embodiments of the present disclosure may include any configuration whereby the sensor information can be communicated with a GUV light source to control operation of the GUV light source based on the sensor information. It will be understood that the GUV light source may be a UV light source (e.g., 100, etc.) as described herein, or may be another light source (e.g., a non-solid state light source, such as an excimer lamp or other conventional UV light source). That is, the operations and components of FIG. 17 may be used with any UV light source, including (but not limited to) the UV light sources described herein.

Benefits of embodiments of the present disclosure may include overall optimization of the operation of GUV illumination systems, including maximization of pathogen disinfection per unit cost. Cost can be reduced, for example, by more effectively operating the illumination devices (e.g., operating the GUV light source at higher intensities for short periods of time), operating the GUV light source when the sensors indicate that value is maximized, and/or by utilizing fewer units to cover a given space (thus reducing cost). Cost can also be reduced by reducing or minimizing the overall time that a given GUV light source is on, i.e., effectively reducing the duty factor. This can extend the lifetime of the GUV light source and thus reduce overall operating cost. Beneficiaries of such improved operation and/or optimization may include both customers, system operators, and also any persons who come into contact with the disinfection technology.

Commercial applications for far-UVC illumination in accordance with embodiments of the present disclosure can include elimination of pathogens from air and/or surfaces in any indoor spaces where humans congregate (e.g., airports, schools, hospitals, inpatient care centers, workplaces, etc.), as well as in transportation vehicles (e.g., subway cars, trains, taxis, airplanes) and agricultural settings (e.g., animal production facilities, meatpacking facilities, indoor greenhouses, etc.).

Additionally the generation of far-UVC light 121′ may have numerous applications beyond germicidal use, which may include (but are not limited to) spectroscopy, optical sensing, detection, etc. In particular, UV light sources configured to provide far-UVC illumination in accordance with embodiments of the present disclosure can be used the detection of trace chemical or biological species in various field environments (air, water, etc.), in which UV fluorescence and Raman spectroscopy are widely used and developed. The use of extremely short wavelength (e.g., in the far-UVC wavelength range) excitation for such applications may be beneficial to each in different ways. For example, the efficiency of Raman scattering may scale inversely with excitation wavelength to the fourth power (1/λ⁴). For fluorescence applications, moving the excitation wavelength further into the UV range can open up a wider spectral range of possible emission, with reduced or minimal background from the excitation wavelength or background light.

Some existing light sources used to generate these (far) UV wavelengths may be expensive, large, may not achieve the required wavelengths, and/or may not be human safe. In contrast, UV light sources in accordance with embodiments of the present disclosure may provide several attributes that may be particularly useful for Raman and/or UV spectroscopy applications, including (but not limited to) (a) small size per unit optical output, (b) low cost, (c) ability to operate in the solar blind region of the visible spectrum (i.e., with emission wavelengths in a spectral range that is free of background noise from the sun), and (d) emission in human safe wavelength ranges. Embodiments described herein can thereby provide new ways of deploying fluorescence and Raman spectroscopy into low cost handheld devices or low cost wall mountable devices that monitor environments in which people are persistently present.

UV light sources according to embodiments of the present disclosure may further generate output light 131 (e.g., SHG/SFG light 121′) over a very narrow bandwidth (e.g., with an emission linewidth or bandwidth of less than about 1 nm, for example, less than about 0.5 nm, or less than about 0.1 nm). In some embodiments, the output light 131 may be emitted from an edge of the output coupling element 130, for example, as a coherent beam. That is, in addition to providing output light 131 in the far-UVC wavelength range (about 200-240 nm), the linewidth of the emission from some embodiments of our invention may be, for example, less than about 0.1 nm, which is far narrower than some conventional light sources. Raman spectroscopy applications, in particular, may benefit from an extremely narrow spectral width for the light source.

Various embodiments have been described herein with reference to the accompanying drawings in which example embodiments are shown. These embodiments may, however, be embodied in different forms and should not be construed as limited to the embodiments set forth herein. Rather, these embodiments are provided so that this disclosure is thorough and complete and fully conveys the inventive concept to those skilled in the art. Various modifications to the example embodiments and the generic principles and features described herein will be readily apparent. In the drawings, the sizes and relative sizes of layers and regions are not shown to scale, and in some instances may be exaggerated for clarity.

The example embodiments are mainly described in terms of particular methods and devices provided in particular implementations. However, the methods and devices may operate effectively in other implementations. Phrases such as “example embodiment”, “one embodiment” and “another embodiment” may refer to the same or different embodiments as well as to multiple embodiments. The embodiments will be described with respect to systems and/or devices having certain components. However, the systems and/or devices may include fewer or additional components than those shown, and variations in the arrangement and type of the components may be made without departing from the scope of the inventive concepts.

The example embodiments will also be described in the context of particular methods having certain steps or operations. However, the methods and devices may operate effectively for other methods having different and/or additional steps/operations and steps/operations in different orders that are not inconsistent with the example embodiments. Thus, the present inventive concepts are not intended to be limited to the embodiments shown, but are to be accorded the widest scope consistent with the principles and features described herein.

It will be understood that when an element is referred to or illustrated as being “on,” “connected,” or “coupled” to another element, it can be directly on, connected, or coupled to the other element, or intervening elements may be present. In contrast, when an element is referred to as being “directly on,” “directly connected,” or “directly coupled” to another element, there are no intervening elements present.

It will also be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

It will also be understood that the term “and/or” as used herein refers to and encompasses any and all possible combinations of one or more of the associated listed items. It will be further understood that the terms “include,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to illustrations that are schematic illustrations of idealized embodiments (and intermediate structures) of the invention. As such, variations from the shapes of the illustrations as a result, for example, of manufacturing techniques and/or tolerances, are to be expected. Thus, the regions illustrated in the figures are schematic in nature and their shapes are not intended to illustrate the actual shape of a region of a device and are not intended to limit the scope of the invention.

Unless otherwise defined, all terms used in disclosing embodiments of the invention, including technical and scientific terms, have the same meaning as commonly understood by one of ordinary skill in the art to which this invention belongs, and are not necessarily limited to the specific definitions known at the time of the present disclosure being described. Accordingly, these terms can include equivalent terms that are created after such time. It will be further understood that terms, such as those defined in commonly used dictionaries, should be interpreted as having a meaning that is consistent with their meaning in the present specification and in the context of the relevant art and will not be interpreted in an idealized or overly formal sense unless expressly so defined herein. All publications, patent applications, patents, and other references mentioned herein are incorporated by reference in their entireties.

Many different embodiments have been disclosed herein, in connection with the above description and the drawings. It will be understood that it would be unduly repetitious and obfuscating to literally describe and illustrate every combination and subcombination of these embodiments. Accordingly, the present specification, including the drawings, shall be construed to constitute a complete written description of all combinations and subcombinations of the embodiments of the present disclosure described herein, and of the manner and process of making and using them, and shall support claims to any such combination or subcombination.

Although the invention has been described herein with reference to various embodiments, it will be appreciated that further variations and modifications may be made within the scope and spirit of the principles of the invention as set forth in the following claims.

1. An ultraviolet (UV) light source, comprising:
a light emitting element that is configured to generate light of a first frequency;
a nonlinear optical element that is configured to receive the light of the first frequency from the light emitting element and generate far-UVC light of a second frequency from the light of the first frequency; and
an output coupling element that is configured to selectively outcouple the far-UVC light from the nonlinear optical element as output light.

2. The UV light source of claim 1, wherein the output coupling element is configured to selectively outcouple the far-UVC light into at least one direction that is different than a direction of propagation of the light of the first frequency to provide the output light,
optionally wherein the output light is substantially free of the light of the first frequency.

3. The UV light source of claim 1, wherein the nonlinear optical element comprises aluminum nitride (AlN), optionally wherein the light emitting element and/or the output coupling element comprise a Group III nitride-based material.

4. The UV light source of claim 1, wherein the nonlinear optical element is or comprises an optical cavity that is at least partially resonant at the first frequency.

5. The UV light source of claim 4, wherein the nonlinear optical element has a ring configuration that defines the optical cavity.

6. The UV light source of claim 4 or 5, wherein the nonlinear optical element comprises a plurality of nonlinear optical elements that are arranged to receive the light of the first frequency from the light emitting element.

7. The UV light source of claim 6, further comprising:
an input coupling element that is configured to receive the light of the first frequency from the light emitting element, wherein the plurality of nonlinear optical elements are arranged along the input coupling element.

8. The UV light source of claim 7, wherein respective ones of the nonlinear optical elements comprise different dimensions and/or materials, and wherein the output coupling element comprises a plurality of output coupling elements that are respectively configured to selectively outcouple the far-UVC light from the respective ones of the nonlinear optical elements.

9. The UV light source of claim 4, wherein the optical cavity includes the light emitting element and the nonlinear optical element therein.

10. The UV light source of claim 9, wherein the optical cavity has a linear shape or a closed curve shape.

11. The UV light source of claim 1, wherein the output coupling element comprises at least one of:
a facet having a refractive index that is configured to selectively outcouple the far-UVC light in a first direction; or
a grating having a diffraction order that is configured to selectively outcouple the far-UVC light in a second direction, different than the first direction.

12. The UV light source of claim 11, wherein the nonlinear optical element and the output coupling element are integrated in a same output element that is configured to outcouple the far-UVC light at a plurality of positions or continuously along a length thereof.

13. The UV light source of claim 11, wherein the UV light source is configured to provide the output light substantially free of phase matching between the light of the first frequency and the far-UVC light of the second frequency.

14. The UV light source of claim 11, wherein at least one of the nonlinear optical element and the output coupling element is configured to provide phase matching between the far-UVC light of the second frequency and the light of the first frequency.

15. The UV light source of claim 1, wherein the light emitting element is a laser comprising a lasing cavity, wherein the laser is configured to generate the light of the first frequency, optionally wherein the laser comprises a Group III nitride-based material.

16. The UV light source of claim 15, wherein the light emitting element further comprises one or more optical resonators that are configured to reflect the light of the first frequency and are arranged at first and second ends of the lasing cavity.

17. The UV light source of claim 15, wherein the nonlinear optical element is configured to receive the light of the first frequency from an intra-cavity portion between first and second ends of the lasing cavity.

18. The UV light source of claim 15, wherein the nonlinear optical element comprises first and second nonlinear optical elements positioned at first and second ends of the lasing cavity, respectively.

19. The UV light source of any of claim 15, further comprising:
a saturable absorber in the lasing cavity and configured to generate the light of the first frequency as a plurality of light pulses at a predetermined pulse repetition frequency and duty factor.

20. The UV light source of claim 1, further comprising:
at least one tuning mechanism that is configured to adjust one or more operating characteristics of the nonlinear element based on the light of the first frequency.

21. The UV light source of claim 1, further comprising:
a monitor element that is configured to measure a property of the output light and generate a feedback signal to a controller that is configured to operate the light emitting element and/or the tuning mechanism.

22. The UV light source of claim 1, further comprising:
a substrate having the light emitting element, the nonlinear optical element, and the output coupling element on a surface thereof,
wherein two or more of the light emitting element, the nonlinear optical element, the output coupling element, or connecting waveguides therebetween overlap in a direction perpendicular to the surface of the substrate.

23. The UV light source of claim 1, wherein the output coupling element comprises a plurality of output coupling elements that are configured to outcouple the far-UVC light in respective directions, to provide the output light with a desired far field pattern.

24. The UV light source of claim 1, further comprising:
a controller; and
one or more sensors configured to detect real-time conditions in an operating environment of the UV light source, and to transmit detection signals indicating the real-time conditions to the controller,
wherein the controller is configured to control operation of the light emitting element based on the detection signals.

25. The UV light source of claim 1, wherein the second frequency comprises a sum of or a harmonic of the first frequency.

26. The UV light source of claim 25, wherein the first frequency corresponds to a first wavelength in a range of about 400 nanometers (nm) to 480 nm, and the second frequency corresponds to a second wavelength in a range of about 200 nm to 240 nm.

27. The UV light source of claim 1, wherein the light emitting element and the nonlinear optical element comprise respective elements that are arranged on a non-native substrate.

28. The UV light source of claim 1, wherein the light emitting element and the nonlinear optical element are integrated in a monolithic structure.

29. The UV light source of claim 27, wherein the UV light source comprises an array including a plurality of the light emitting element and the nonlinear optical element.

30. A light source, comprising:
a monolithic structure comprising a light emitting element that is configured to generate light of a first frequency, and a nonlinear optical element that is configured to receive the light of the first frequency from the light emitting element and generate light of a second frequency from the light of the first frequency.

31.-41. (canceled)

42. An ultraviolet (UV) light source, comprising:
a light emitting element that is configured to generate light of a first frequency; and
a nonlinear optical element comprising aluminum nitride (AlN) that is configured to receive the light of the first frequency from the light emitting element and generate UVC light of a second frequency from the light of the first frequency.

43.-51. (canceled)

52. An ultraviolet (UV) light source, comprising:
a light emitting element that is configured to generate light of a first frequency; and
an optical cavity comprising a nonlinear optical element that is configured to receive the light of the first frequency from the light emitting element and generate UVC light of a second frequency from the light of the first frequency,
wherein the optical cavity is at least partially resonant at the first frequency.

53.-61. (canceled)

62. A light source, comprising:
a light emitting element that is configured to generate light of a first frequency; and
a nonlinear optical output coupling element that is configured to receive the light of the first frequency from the light emitting element, generate light of a second frequency from the light of the first frequency, and outcouple the light of the second frequency as output light at a plurality of positions or continuously along a length thereof.

63.-70. (canceled)
